(19) United States
(12) Patent Application Publication
(10) Pub. No.: US 2018/0200233 A1
(43) Pub. Date: Jul. 19, 2018

(54) COMPOSITIONS FOR PREVENTING CANCERS ASSOCIATED WITH HUMAN PAPILLOMA VIRUSES

(71) Applicant: Loma Linda University, Loma Linda, CA (US)

(72) Inventors: Penelope Duerksen-Hughes, Redlands, CA (US); Chung-Hsiang Yuan, Loma Linda, CA (US); Maria Filippova, Riverside, CA (US); John Krstenansky, Redlands, CA (US)

(21) Appl. No.: 15/744,711

(22) PCT Filed: Jul. 12, 2016
(86) PCT No.: PCT/US16/41931

§ 371 Date: Jan. 12, 2018

Related U.S. Application Data
(60) Provisional application No. 62/193,500, filed on Jul. 16, 2015.

(51) Int. Cl. A61K 31/437; A61K 45/06; A61P 35/00

(52) U.S. Cl. CPC A61K 31/437; A61P 35/00; A61K 45/06

(57) ABSTRACT

Compositions and methods of treatment are provided for preventing cancers caused by high-risk human papilloma viruses (HPV). Cancers amenable to prevention include cervical cancer, head cancers, neck cancers, and oral cancers. The compositions block interaction between HPV 16 E6, one of two major viral oncogenes, and its partners, thereby resensitizing HPV positive cells to apoptosis.

### INCORPORATION BY REFERENCE TO RELATED APPLICATIONS

Any and all priority claims identified in the Application Data Sheet, or any correction thereto, are hereby incorporated by reference under 37 CFR 1.57. This application claims the benefit of U.S. Provisional Application No. 62/193,500, filed Jul. 16, 2015. The aforementioned application is incorporated by reference herein in its entirety, and is hereby expressly made a part of this specification.

### STATEMENT REGARDING FEDERALLY SPONSORED R&D

This invention was made with United States Government Support under National Institutes of Health Grant No. R21 NS073059. The United States Government has certain rights in this invention.

### FIELD

Compositions and methods of treatment are provided for treating or preventing cancers caused by high-risk human papilloma viruses (HPV). Cancers amenable to prevention include cervical cancer, head cancers, neck cancers, and oral cancers. The compositions block interaction between HPV 16 E6, one of two major viral oncogenes, and its partners, thereby resensitizing HPV positive cells to apoptosis or other cell death processes.

### BACKGROUND

High-risk types of human papillomavirus (HPV), especially types 16 and 18, are the causative agents of nearly all cases of human cervical cancer, in addition to up to 70% of head and neck cancers (HNC) (see, e.g., Chaturvedi, A. K., et al., Human papillomavirus and rising oropharyngeal cancer incidence in the United States. J Clin Oncol, 2011. 29(32): p. 4294-301). Although the overall incidence of HNC has stabilized during the last decade, the incidence of HPV-associated cases, especially of oropharyngeal squamous cell carcinoma, has dramatically increased (see. e.g., Adams, A. K., T. M. Wise-Draper, and S. I. Wells, Human papillomavirus induced transformation in cervical and head and neck cancers. Cancers (Basel), 2014. 6(3): p. 1793-820). HPVs are small, double stranded DNA viruses that infect epithelial tissues. The HPV-encoded oncogenes E6 and E7 are responsible for cellular immortalization and transformation, and consequently, for the development of HPV-associated cancer. Although E7 is best known for the inactivation of Rb, E6 accelerates the degradation of several molecules involved in apoptosis.

Two HPV vaccines, Gardasil (MSD MSD, Merck, Kenilworth, N.J., USA) and Cervarix (GSK, Glaxo SmithKline, London, UK), have been approved and are currently in use for the prevention of HPV infection. However, they offer no benefit to an individual who has already been infected, and only protect against two of the 15 types of high-risk viruses, HPV-16 and -18. Surgical and ablative techniques are used to remove developed tumors; however, these approaches are invasive and cytodestructive, and lesions frequently recur following treatment. Chemotherapy, utilizing agents such as cisplatin and doxorubicin, has also been used to treat cervical cancer, but with mixed results (see, e.g., Bonomi, P., et al., Randomized trial of three cisplatin dose schedules in squamous-cell carcinoma of the cervix: a Gynecologic Oncology Group study. Journal of clinical oncology: official journal of the American Society of Clinical Oncology, 1985. 3(8): p. 1079-85; Thigpen, T., et al., Cis-platinum in treatment of advanced or recurrent squamous cell carcinoma of the cervix: a phase II study of the Gynecologic Oncology Group. Cancer, 1981. 48(4): p. 899-903; Thigpen, J. T., et al., A randomized comparison of a rapid versus prolonged (24 hr) infusion of cisplatin in therapy of squamous cell carcinoma of the uterine cervix: a Gynecologic Oncology Group study. Gynecologic oncology, 1989. 32(2): p. 198-202; Scatchard, K., et al., Chemotherapy for metastatic and recurrent cervical cancer. Cochrane Database Syst Rev, 2012. 10: p. CD006469; Lee, T. Y., et al., Promising treatment results of adjuvant chemotherapy following radical hysterectomy for intermediate risk stage 1B cervical cancer. Obstet Gynecol Sci, 2013. 56(1): p. 15-21; and Minotti, G., et al., Anthracyclines: molecular advances and pharmacologic developments in antitumor activity and cardiotoxicity. Pharmacological reviews, 2004. 56(2): p. 185-229).

As researchers and clinicians have worked to move beyond these relatively non-specific and toxic agents, reagents that activate the tumor necrosis factor-related apoptosis-inducing ligand (TRAIL)-mediated, extrinsic apoptotic pathway have garnered considerable interest owing to their promise in the treatment of several types of tumors (see, e.g., Buchsbaum, D. J., et al., Antitumor efficacy of TRA-8 anti-DR5 monoclonal antibody alone or in combination with chemotherapy and/or radiation therapy in a human breast cancer model. Clin Cancer Res, 2003. 9(10 Pt 1): p. 3731-41; Younes, A. and M. E. Kadin, Emerging applications of the tumor necrosis factor family of ligands and receptors in cancer therapy. J Clin Oncol, 2003. 21(18): p. 3526-34; Ichikawa, K., et al., Tumoricidal activity of a novel anti-human DR5 monoclonal antibody without hepatocyte cytotoxicity. Nat Med, 2001. 7(8): p. 954-60; Chuntharapai, A., et al., Isotype-dependent inhibition of tumor growth in vivo by monoclonal antibodies to death receptor 4. J Immunol, 2001. 166(8): p. 4891-8; Tanaka, S., et al., Expression and antitumor effects of TRAIL in human cholangiocarcinoma. Hepatology, 2000. 32(3): p. 523-7; Bellail, A. C., et al., TRAIL agonists on clinical trials for cancer therapy: the promises and the challenges. Rev Recent Clin Trials, 2009. 4(1): p. 34-41; El-Zawahry, A., J. McKillop, and C. Voelkel-Johnson, Doxorubicin increases the effectiveness of Apo2L/TRAIL for tumor growth inhibition of prostate cancer xenografts. BMC Cancer, 2005. 5: p. 2; Mom, C. H., et al., Mapatumumab, a fully human agonistic monoclonal antibody that targets TRAIL-R1, in combination with gemcitabine and cisplatin: a phase I study. Clin Cancer Res, 2009. 15(17): p. 5584-90; and Naka, T., et al., Effects of tumor necrosis factor-related apoptosis-inducing ligand alone and in combination with chemotherapeutic agents on patients' colon tumors grown in SCID mice. Cancer Res, 2002. 62(20): p. 5800-6).

Unfortunately, therapies that function by activating apoptosis, including those based on TRAIL, cisplatin and doxorubicin, are handicapped in their ability to effectively treat human papillomavirus (HPV)-associated malignancies because high-risk E6 proteins subvert both the extrinsic and intrinsic apoptotic pathways. E6 proteins from high-risk types of human papillomavirus are well known for their ability to mediate the rapid degradation of p53 (e.g., see Huibregtse, J. M., M. Scheffner, and P. M. Howley, A cellular protein mediates association of p53 with the E6 oncoprotein of human papillomavirus types 16 or 18. Embo J, 1991. 10(13): p. 4129-35; Scheffner, M., et al., The HPV-16 E6 and E6-AP complex functions as a ubiquitin-protein ligase in the ubiquitination of p53. Cell, 1993. 75(3): p. 495-505; Scheffner, M., et al., The E6 oncoprotein encoded by human papillomavirus types 16 and 18 promotes the degradation of p53. Cell, 1990. 63(6): p. 1129-36; and Huibregtse, J. M., M. Scheffner, and P. M. Howley, A cellular protein mediates association of p53 with the E6 oncoprotein of human papillomavirus types 16 or 18. The EMBO journal, 1991. 10(13): p. 4129-35), an important mediator of intrinsic apoptotic pathways, thereby increasing the growth and survival of transformed cells (see, e.g., Crook, T., J. A. Tidy, and K. H. Vousden, Degradation of p53 can be targeted by HPV E6 sequences distinct from those required for p53 binding and trans-activation. Cell, 1991. 67(3): p. 547-56; Lechner, M. S. and L. A. Laimins, Inhibition of p53 DNA binding by human papillomavirus E6 proteins. Journal of virology, 1994. 68(7): p. 4262-73). E6 also interacts with other partner proteins, a number of which participate in extrinsic, receptor-mediated apoptosis. For example, it was found that HPV 16 E6 binds to and inactivates a number of molecules involved in these pathways, including TNF R1 (Tungteakkhun, S. S., et al., The full-length isoform of human papillomavirus 16 E6 and its splice variant E6* bind to different sites on the procaspase 8 death effector domain. J Virol, 2010. 84(3): p. 1453-63), Fas-associated protein with death domain (FADD) (Filippova, M., L. Parkhurst, and P. J. Duerksen-Hughes, The human papillomavirus 16 E6 protein binds to Fas-associated death domain and protects cells from Fas-triggered apoptosis. J Biol Chem, 2004. 279(24): p. 25729-44) and caspase 8 (Filippova, M., et al., The large and small isoforms of human papillomavirus type 16 E6 bind to and differentially affect procaspase 8 stability and activity. J Virol, 2007. 81(8): p. 4116-29; Garnett, T. O., M. Filippova, and P. J. Duerksen-Hughes, Accelerated degradation of FADD and procaspase 8 in cells expressing human papilloma virus 16 E6 impairs TRAIL-mediated apoptosis. Cell Death Differ, 2006. 13(11): p. 1915-26). As a result, engagement of either the extrinsic or the intrinsic cell death pathways fails to result in the transduction of the intended death signal because the mediator molecules—p53 in the case of the intrinsic pathway, and FADD and caspase 8 in the case of the extrinsic pathway—are missing. Therefore, if any of these cell death-inducing signaling pathways are to be used as effective tools for the elimination of HPV-associated malignancies, it will be necessary to restore the missing signaling molecules.

Myricetin has been identified as a compound that can inhibit the E6/caspase 8 interaction in vitro (Yuan, C. H., et al., Small molecule inhibitors of the HPV16-E6 interaction with caspase 8. Bioorganic & medicinal chemistry letters, 2012. 22(5): p. 2125-9). Unfortunately, myricetin is known to also inhibit a number of cellular proteins including several tyrosine kinases, and its structure makes modification for drug development difficult.

### BRIEF DESCRIPTION OF THE DRAWINGS

The details of the present disclosure, both as to its structure and operation, may be understood in part by study of the accompanying drawings, in which like reference numerals refer to like parts. The drawings are not necessarily to scale, emphasis instead being placed upon illustrating the principles of the disclosure.

FIG. 1 provides structures of compounds tested for activity.

FIGS. 2A-C are graphs providing data regarding inhibition of protein-protein interactions by 3-(1H-indol-1yl)propan-1-amine (1), three benzimidazole derivatives (2-4), and spinacine (5). Compounds, at the indicated concentrations (1.4 μM to 3.2 mM) were tested using a bead-based screening assay for their ability to inhibit three different protein/protein interactions: (FIG. 2A) GST-E6/His-caspase 8; (FIG. 2B) GST-E6/HisE6AP; and (FIG. 2C) GST-caspase 8/His-caspase 8. Binding in the presence of 1.4 μM of the test compound was set at 100%. Experiments were performed in triplicate, and error bars indicate the standard deviation.

FIGS. 3A-E are graphs providing data regarding resistance of HPV⁺ SiHa cells to TRAIL-, cisplatin- and doxocycline-induced apoptosis, in the presence and absence of small molecules as disclosed herein. (FIG. 3A) HPV⁺ SiHa cells are resistant to TRAIL-induced apoptosis as compared to the human osteosarcoma cell line U2OS. Cells (2×10⁴ cells per well) were seeded into a 96-well plate, allowed to incubate overnight, and then treated with indicated concentration of TRAIL in the presence of cycloheximide (5 μg/ml). The viability of cells was measured after overnight incubation using the MTT assay; the viability of cells untreated with TRAIL was set at 100% for each group. (FIG. 3B) The indicated concentrations of three small molecules were added to SiHa cells, and an MTT assay was preformed after overnight incubation. The viability of cells untreated with small molecules was set as 100%. (FIG. 3C) The indicated concentrations of myricetin or spinacine were added 4 h prior to TRAIL (100 ng/ml) in the presence of cycloheximide (5 μg/ml) and incubated at 37° C. overnight. The viability of cells untreated with small molecules was set at 100%. (FIG. 3D and FIG. 3E) SiHa cells (2×10⁴ cells per well) were seeded into a 96-well plate and allowed to incubate overnight. 100 μM of myricetin and 50 μM of spinacine were added 4 h prior to either (FIG. 3D) doxorubicin or (FIG. 3E) cisplatin. Cell viability was measured after overnight incubation using the MTT assay, and the viability of cells untreated with either myricetin or spinacine was set at 100%. Experiments were performed in triplicate, and error bars indicate the standard deviation.

FIGS. 4A-C are graphs showing that myricetin and spinacine (5) increased caspase 3/7 activity in SiHa cells following treatment with TRAIL and chemotherapy drugs. SiHa cells (2×10⁴ cells per well) were seeded into a 96-well plate and allowed to incubate overnight, then pre-treated with 100 μM of myricetin or 50 μM of spinacine for 4 h. (FIG. 4A) TRAIL (100 ng/ml), along with cycloheximide (5 μg/ml), (FIG. 4B) cisplatin (50 μM), or (FIG. 4C) doxorubicin (2 μM) were added respectively. Caspase 3/7 activity was measured after 0, 1.5, 3, and 6 hours using the CellTiter-Glo assay. Activity at 0 h of treatment was set at 100% for each group. Experiments were performed in triplicate, and error bars indicate the standard deviation.

FIGS. 5A-B are graphs showing myricetin and spinacine (5) re-sensitized HPV⁺, but not HPV⁻, cells to treatment with TRAIL. SiHa (2×10⁴ cells per well) and C33A cells (1×10⁴ cells per well) were seeded into a 96-well plate and allowed to incubate overnight, then cells were pre-treated in the presence or absence of myricetin (100 μM) (FIG. 5A) or spinacine (50 μM) (FIG. 5B) for 4 h. The indicated concentration of TRAIL, along with cycloheximide (5 μg/ml) was added and cells were allowed to incubate overnight. Cell viability was measured by MTT assay, and the viability of cells untreated with TRAIL was set at 100%. Experiments were performed in triplicate, and error bars indicate the standard deviation.

FIGS. 6A-B is an immunoblot and FIG. 6B is a graph showing that treatment with myricetin and spinacine (5) increased cellular levels of caspase 8 and p53. (FIG. 6A) SiHa cells (1×10⁶ per well) were seeded into a 6-well plate and allowed to incubate overnight. The indicated concentrations of myricetin and spinacine were added, then cells were incubated for 24 h. Cells were then washed in 1×PBS, then harvested and the resulting level of caspase 8 was measured by immunoblot. (FIG. 6B) SiHa and C33A cells (1×10⁶ per well) were seeded into a 6-well plate and allowed to incubate overnight. 200 μM of myricetin and 100 μM spinacine were added together with 4 μg/ml mitomycin C and incubated for 24 h. The resulting level of p53 was measured by ELISA, and the level of p53 found in untreated cells was set at 100%. Experiments were performed in triplicate, and error bars indicate the standard deviation.

FIGS. 7A-E are graphs showing myricetin and spinacine (5) re-sensitized HPV⁺, but not HPV⁻, head and neck cancer cell lines to treatment with TRAIL. (FIG. 7A) Both HPV⁻ and HPV⁺ HN cancer cell lines display resistance to TRAIL treatment. HPV⁻ (#84) (Myricetin—FIG. 7B; Spinacine—FIG. 7C) and HPV⁺ (#90) (FIG. 7D; Spinacine—FIG. 7E) head and neck cancer cell lines (2×10⁴ cells per well) were seeded into 96-well plates and allowed to incubate overnight, then cells were pre-treated with myricetin (0-200 μM) or spinacine (0-100 μM) for 4 h. 50 μM of TRAIL, along with cycloheximide (5 μg/ml) was added and cells were allowed to incubate overnight. Cell viability was measured by the MTT assay, and the viability of cells untreated with small molecules was set at 100%. Experiments were performed in triplicate, and error bars indicate the standard deviation.

FIGS. 8A-B includes graphs showing myricetin and spinacine (5) re-sensitized HPV⁺, but not HPV⁻, HNSCC cell lines to treatment with doxorubicin. One HPV⁻ (FIG. 8A; #84) and one HPV⁺ (FIG. 8B; #90) head and neck cancer cell line (2×10⁴ cells per well) were seeded into 96-well plates and allowed to incubate overnight, then cells were pre-treated with myricetin or spinacine (0-12.5 μM) for 4 h. 2 μM of doxorubicin was added and cells were allowed to incubate overnight. Cell viability was measured by MTT assay, and the viability of cells untreated with small molecules was set at 100%. Experiments were performed in triplicate, and error bars indicate the standard deviation.

FIG. 9 is a graph showing spinacine is able to bind to GST-E6. N<1 could indicate that the protein concentration is too low or the spinacine concentration is too high. An N of about 0.5 indicates that there are likely two molecules of spinacine that bind to one molecule of E6.

FIG. 10 is a graph showing activity of D,L-spinacine dehydrate (10-1); D-spinacine hydrochloride (10-2); L-spinacine hydrochloride (10-3); spinacine (10-4); and spinacine (10-5) in binding GST-E6/His-caspase 8.

FIG. 11 is a graph showing activity of D,L-spinacine dehydrate (10-1); D-spinacine hydrochloride (10-2); L-spinacine hydrochloride (10-3); spinacine (10-4); and spinacine (10-5) in binding GST-E6/His-E6AP.

FIGS. 12A-F are NMR data for a sample of D-spinacine.

### SUMMARY OF THE INVENTION

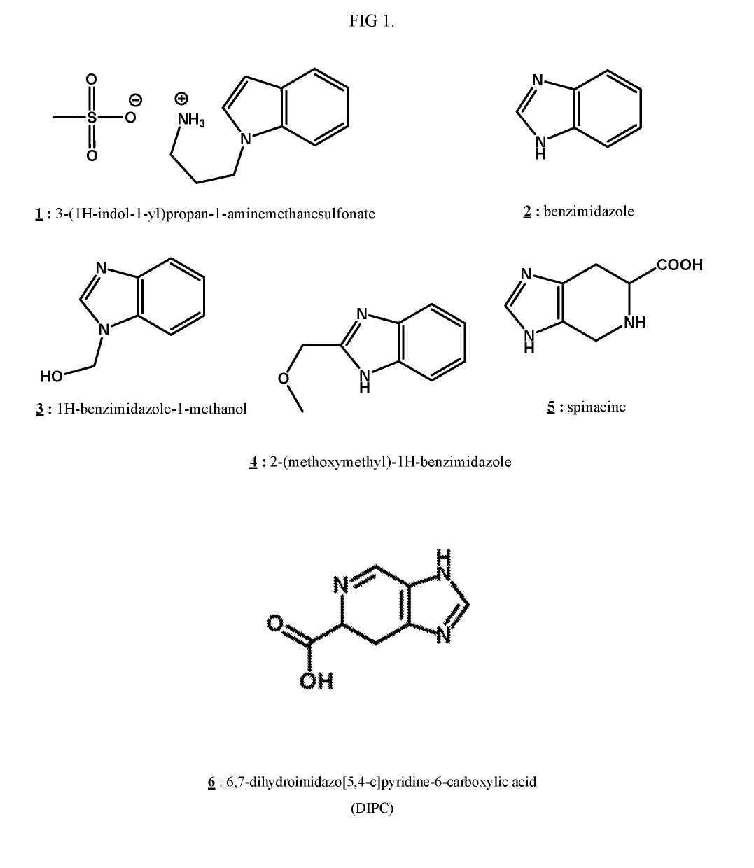

The identification of additional, more tractable inhibitors of the E6/procaspase 8 interaction is desirable. One such compound, spinacine (5; see FIG. 1), an unnatural amino acid that is the product of the action of formaldehyde on histidine, has been determined to inhibit specific interactions between E6 and its partner proteins. It was found that both myricetin and spinacine can re-sensitize HPV+ cells to apoptosis or other cell death pathways triggered by inducers such as TRAIL, cisplatin, and doxorubicin which increase caspase 3/7 activity and thus restore the level of apoptotic proteins in HPV+ cells. Accordingly, methods of inhibiting E6 functions by use of spinacine are provided, which may provide effective therapeutic approaches for the treatment and/or prevention of HPV-mediated cancers.

In a first aspect, which is generally applicable (i.e. independently combinable with any of the aspects or embodiments identified herein), a method of treating or preventing a cancer caused by a human papilloma virus is provided, comprising administering to a subject in need thereof an effective amount of spinacine.

In an embodiment of the first aspect, which is generally applicable (i.e., independently combinable with any of the aspects or embodiments identified herein), the spinacine is D,L-spinacine.

In an embodiment of the first aspect, which is generally applicable (i.e., independently combinable with any of the aspects or embodiments identified herein), the spinacine is D-spinacine.

In an embodiment of the first aspect, which is generally applicable (i.e., independently combinable with any of the aspects or embodiments identified herein), the spinacine is L-spinacine.

In an embodiment of the first aspect, which is generally applicable (i.e., independently combinable with any of the aspects or embodiments identified herein), the method further comprises administering an effective amount of at least one chemotherapeutic agent.

In an embodiment of the first aspect, which is generally applicable (i.e., independently combinable with any of the aspects or embodiments identified herein), the method further comprises administering an effective amount of at least one chemotherapeutic agent selected from the group consisting of cisplatin and doxorubicin.

In an embodiment of the first aspect, which is generally applicable (i.e., independently combinable with any of the aspects or embodiments identified herein), the method comprises administering an effective amount of a combination of 6,7-dihydroimidazo[5,4-c]pyridine-6-carboxylic acid and spinacine.

In an embodiment of the first aspect, which is generally applicable (i.e., independently combinable with any of the aspects or embodiments identified herein), the method further comprises administering an effective amount of at least one TNF superfamily ligand selected from the group consisting of 4-1BB Ligand/TNFSF9, APRIL/TNFSF13, BAFF/BLyS/TNFSF13B, CD27 Ligand/TNFSF7, CD30 Ligand/TNFSF8, CD40 Ligand/TNFSF5, EDA/Ectodysplasin, EDA-A2/Ectodysplasin A2, Fas Ligand/TNFSF6, GITR Ligand/TNFSF18, LIGHT/TNFSF14, Lymphotoxin, Lymphotoxin beta/TNFSF3, OX40 Ligand/TNFSF4, TL1A/TNFSF15, TNF-alpha, Lymphotoxin-alpha/TNF-beta, TRAIL/TNFSF10, TRANCE/TNFSF11/RANK L, TWEAK/TNFSF12, and EDA-A 1/Ectodysplasin A1.

In an embodiment of the first aspect, which is generally applicable (i.e., independently combinable with any of the aspects or embodiments identified herein), the subject is mammalian.

In an embodiment of the first aspect, which is generally applicable (i.e., independently combinable with any of the aspects or embodiments identified herein), the subject is human.

In an embodiment of the first aspect, which is generally applicable (i.e., independently combinable with any of the aspects or embodiments identified herein), the cancer is selected from the group consisting of cervical cancer, head cancers, neck cancers, oral cancers, oropharyngeal cancer, anal cancer, vaginal cancer, vulvar cancer, and penile cancer.

In an embodiment of the first aspect, which is generally applicable (i.e., independently combinable with any of the aspects or embodiments identified herein), the spinacine is administered parentally, intravenously, or transmebranally.

In a second aspect, which is generally applicable (i.e., independently combinable with any of the aspects or embodiments identified herein), a method of blocking interaction between a HPV 16 E6 viral oncogene and a partner, thereby resensitizing a high-risk human papilloma virus positive cell to cell death mechanisms including apoptosis, is provided, comprising contacting the cell with an effective amount of spinacine.

In an embodiment of the second aspect, which is generally applicable (i.e., independently combinable with any of the aspects or embodiments identified herein), the cell is mammalian.

In an embodiment of the second aspect, which is generally applicable (i.e., independently combinable with any of the aspects or embodiments identified herein), the cell is human.

In an embodiment of the second aspect, which is generally applicable (i.e., independently combinable with any of the aspects or embodiments identified herein), the cell is in vivo.

In an embodiment of the second aspect, which is generally applicable (i.e., independently combinable with any of the aspects or embodiments identified herein), the cell is ex vivo.

In a third aspect, a pharmaceutical composition is provided comprising spinacine and a pharmaceutically acceptable excipient.

In a fourth aspect, a method of treating or preventing a cancer caused by a human papilloma virus is provided, comprising administering to a subject in need thereof an effective amount of 6,7-dihydroimidazo[5,4-c]pyridine-6-carboxylic acid.

In an embodiment of the fourth aspect, which is generally applicable (i.e., independently combinable with any of the aspects or embodiments identified herein), the method further comprises administering an effective amount of at least one chemotherapeutic agent.

In an aspect of the fourth embodiment, which is generally applicable (i.e., independently combinable with any of the aspects or embodiments identified herein), the method further comprises administering an effective amount of at least one chemotherapeutic agent selected from the group consisting of cisplatin and doxorubicin.

In an aspect of the fourth embodiment, which is generally applicable (i.e., independently combinable with any of the aspects or embodiments identified herein), the method further comprises administering an effective amount of at least one TNF superfamily ligand selected from the group consisting of 4-1BB Ligand/TNFSF9, APRIL/TNFSF13, BAFF/BLyS/TNFSF13B, CD27 Ligand/TNFSF7, CD30 Ligand/TNFSF8, CD40 Ligand/TNFSF5, EDA/Ectodysplasin, EDA-A2/Ectodysplasin A2, Fas Ligand/TNFSF6, GITR Ligand/TNFSF18, LIGHT/TNFSF14, Lymphotoxin, Lymphotoxin beta/TNFSF3, OX40 Ligand/TNFSF4, TL1A/TNFSF15, TNF-alpha, Lymphotoxin-alpha/TNF-beta, TRAIL/TNFSF10, TRANCE/TNFSF1/RANK L, TWEAK/TNFSF12, and EDA-A1/Ectodysplasin A1.

In an aspect of the fourth embodiment, which is generally applicable (i.e., independently combinable with any of the aspects or embodiments identified herein), the subject is mammalian.

In an aspect of the fourth embodiment, which is generally applicable (i.e., independently combinable with any of the aspects or embodiments identified herein), the subject is human.

In an aspect of the fourth embodiment, which is generally applicable (i.e., independently combinable with any of the aspects or embodiments identified herein), the cancer is selected from the group consisting of cervical cancer, head cancers, neck cancers, oral cancers, oropharyngeal cancer, anal cancer, vaginal cancer, vulvar cancer, and penile cancer.

In an aspect of the fourth embodiment, which is generally applicable (i.e., independently combinable with any of the aspects or embodiments identified herein), the compound is administered parentally, intravenously, or transmebranally.

In a fifth embodiment, a method of blocking interaction between a HPV 16 E6 viral oncogene and a partner, thereby resensitizing a high-risk human papilloma virus positive cell to cell death inducing processes, is provided, comprising contacting the cell with an effective amount of 6,7-dihydroimidazo[5,4-c]pyridine-6-carboxylic acid.

In an aspect of the fifth embodiment, which is generally applicable (i.e., independently combinable with any of the aspects or embodiments identified herein), the cell is mammalian.

In an aspect of the fifth embodiment, which is generally applicable (i.e., independently combinable with any of the aspects or embodiments identified herein), the cell is human.

In an aspect of the fifth embodiment, which is generally applicable (i.e., independently combinable with any of the aspects or embodiments identified herein), the cell is ex vivo.

In a sixth embodiment, a pharmaceutical composition comprising 6,7-dihydroimidazo[5,4-c]pyridine-6-carboxylic acid and a pharmaceutically acceptable excipient is provided.

Any of the features of an aspect of the first through sixth embodiments is applicable to all aspects and embodiments identified herein. Moreover, any of the features of an aspect of the first through sixth embodiments is independently combinable, partly or wholly with other aspects described herein in any way, e.g., one, two, or three or more aspects may be combinable in whole or in part. Further, any of the features of an aspect of the first through sixth embodiments may be made optional to other aspects or embodiments.

### DETAILED DESCRIPTION

The following description and examples illustrate embodiments of the present invention in detail. Those of skill in the art will recognize that there are numerous variations and modifications of this invention that are encompassed by its scope. Accordingly, the description of an embodiment should not be deemed to limit the scope of the present invention. The illustrative embodiments described in the detailed description, drawings, and claims are not meant to be limiting. The teachings herein can be applied in a multitude of different ways, including for example, as defined and covered by the claims. It should be apparent that the aspects herein may be embodied in a wide variety of forms and that any specific structure, function, or both being disclosed herein is merely representative. Based on the teachings herein one skilled in the art should appreciate that an aspect disclosed herein may be implemented independently of any other aspect and that two or more of these aspects may be combined in various ways. For example, a composition may be utilized or a method may be practiced by one of skill in the art using any reasonable number or combination of the aspects set forth herein. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and made part of this disclosure. It is to be understood that the disclosed embodiments are not limited to the examples described below, as other embodiments may fall within disclosure and the claims.

The terms “pharmaceutically acceptable salts” and “a pharmaceutically acceptable salt thereof” as used herein are broad terms, and are to be given their ordinary and customary meaning to a person of ordinary skill in the art (and are not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl esters, and protected derivatives can also be suitable for use in compositions and methods of various embodiments. While it may be possible to administer the compounds of the various embodiments in the form of pharmaceutically acceptable salts, it is generally preferred to administer the compounds in neutral form.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z a mixture thereof.

Likewise, it is understood that, in any compound described, all tautomeric forms are also intended to be included. For example all tautomers of phosphate groups are intended to be included. Furthermore, all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

High-risk HPVs cause several types of cancer. Virtually all cases of cervical cancer are caused by HPV, and just two HPV types, 16 and 18, are responsible for about 70 percent of all cases. About 95 percent of anal cancers are caused by HPV. Most of these are caused by HPV type 16. About 70 percent of oropharyngeal cancers (cancers of the middle part of the throat, including the soft palate, the base of the tongue, and the tonsils) are caused by HPV. In the United States, more than half of cancers diagnosed in the oropharynx are linked to HPV type 16). HPV causes about 65 percent of vaginal cancers, 50 percent of vulvar cancers, and 35 percent of penile cancers. Most of these are caused by HPV type 16.

High-risk human papillomaviruses (HR-HPVs) cause nearly all cases of cervical cancer, as well as approximately 30% of head and neck cancers. HPV 16 E6, one of two major viral oncogenes, protects cells from apoptosis by binding to and accelerating the degradation of several proteins important in apoptotic signaling, including caspase 8 and p53. Blocking the interactions between HPV E6 and its partners using small molecules, HPV⁺ cells can be re-sensitized to apoptosis and other cell death inducing processes. Several compounds having such activity were identified and tested for dose-dependency and specificity in vitro, and for toxicity in a cell-based assay. Myricetin, a flavonol, and spinacine, an imidazole amino acid derivative of histidine clearly inhibited the ability of E6 to bind in vitro to both caspase 8 and E6AP, the protein that mediates p53 degradation. In addition, both compounds were able to increase the level of caspase 8 and p53 in SiHa cervical cancer cells, resulting in an increase of caspase 3/7 activity. Finally, both myricetin and spinacine sensitized HPV⁺ cervical and⁺ oral cancer cells, but not HPV⁻ cervical and oral cancer cells, to apoptosis induced by the cancer-specific ligand TRAIL, as well as the chemotherapeutic agents doxorubicin and cisplatin. New therapies based offer an improved treatment for HPV⁺ cancer patients.

Spinacine has been demonstrated to be a safe, well-tolerated compound. See, e.g., Galli et al., “Evaluation of the Oral Toxicity of Spinacine Hydrochloride in a 13-week Study in Rats” Fd Chem. Toxic. Vol 27, No. 10, pp. 651-656, 1989. Its antiviral activity has been investigated (see e.g., DE 3521303 A1), as well as its antimicrobial activity (see, e.g., Preusser, H. J. “Antimicrobial activity of alkaloids from amphibian venoms and effects on the ultrastructure of yeast cells”, Toxicon., Vol. 13, Issue 4, pp 285-9, 1975), activity as an inhibitor of gamma-aminobutyric acid capture (see, e.g., Boreisha, I. K., Khimiko-Farmatsevticheskii Zhurnal, Vol. 22, Issue 1, pages 20-23, 1988. A synthetic route has been published. See, e.g., Biochemische Zeitschrift, Vol. 49, pp. 173, “Synthetic Alkaloids from Tyrosine Tryptophane and Histidine”, 1913), and its antinausea activity (see, e.g., U.S. Pat. No. 5,262,537). Spinacine can also be derived from Panax ginseng. See, e.g., Han et al., “Spinacine from Panax ginseng”, Archives of Pharmacal Research, Vol. 10, Issue 4, pp 258-259, 1987

Imidazole Derivatives Specifically Inhibit the Interaction of HPV E6 with Caspase 8

HPV E6 binds to caspase 8 (Garnett, T. O., M. Filippova, and P. J. Duerksen-Hughes, Accelerated degradation of FADD and procaspase 8 in cells expressing human papilloma virus 16 E6 impairs TRAIL-mediated apoptosis. Cell death and differentiation, 2006. 13(11): p. 1915-26), and myricetin can block the E6/caspase 8 interaction in vitro (Yuan, C. H., et al., Small molecule inhibitors of the HPV16-E6 interaction with caspase 8. Bioorganic & medicinal chemistry letters, 2012. 22(5): p. 2125-9). A bead-based assay based on AlphaScreen™ technology (Perkin-Elmer) is used to identify inhibitors of the E6/caspase 8 interaction (Yuan, C. H., et al., Small molecule inhibitors of the HPV16-E6 interaction with caspase 8. Bioorganic & medicinal chemistry letters, 2012. 22(5): p. 2125-9). The ActiProbe 2K (2,000 compounds) library was screened using the same approach. In the primary screen, 118 (5.9%) compounds demonstrated an ability to inhibit the E6/caspase 8 interaction. 79 of these 118 compounds presented EC₅₀ values lower than 10 μM, and were therefore chosen for further analysis. 23 of these 79 compounds also demonstrated specific inhibition of the E6/caspase 8 interaction, in that they were unable to block formation of the caspase 8/caspase 8 homodimer (counter-screen). These 23 compounds represented three different chemical classes, of which the imidazoles and the related benzimidazoles showed the best inhibition of E6/caspase 8 binding. Analysis of the structures and physical/chemical characteristics of those molecules led to the selection of five additional compounds: 3-(1H-indol-1-yl)propan-1-amine methanesulfonate (1), benzimidazole (2), 1H-benzimidazole-1-methanol (3), 2-(methoxymethyl)-1H-benzimidazole (4), and spinacine (5) for further study (FIG. 1).

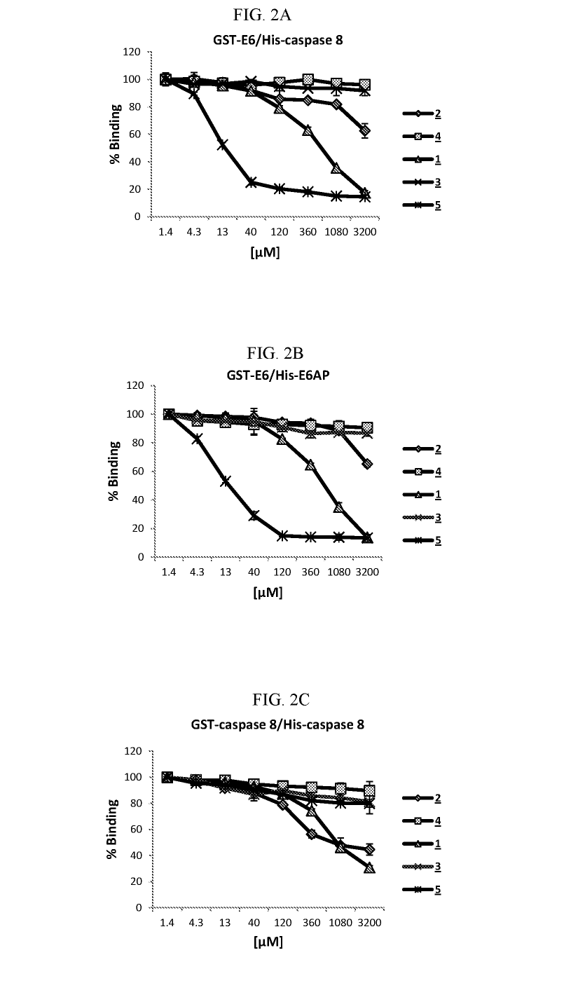

To determine the dose-responsiveness of these five compounds, variable concentrations of those small molecules were first tested for their ability to inhibit E6/caspase binding. The results showed that only compounds 1, 2 and 5 demonstrated dose-dependent inhibition of E6/caspase 8 binding (FIG. 2A). These molecules, which had been selected for their ability to interfere with E6/caspase 8 binding, were then tested to determine if they could also affect other interactions of E6, such as its binding to E6-associated protein (E6AP). The E6/E6AP interaction is required for the E6-mediated acceleration of p53 degradation (Huibregtse, J. M., M. Scheffner, and P. M. Howley, A cellular protein mediates association of p53 with the E6 oncoprotein of human papillomavirus types 16 or 18. The EMBO journal, 1991. 10(13): p. 4129-35). The ability of these five compounds to inhibit E6/E6AP binding was tested, and it was found that compounds 1, 2 and 5, the same agents that had inhibited E6/caspase 8 binding, could also inhibit the E6/E6AP interaction, and could do so nearly as well as they had inhibited the E6/caspase 8 interaction (FIGS. 2A, 2B).

To ask whether this inhibition was specific, a counter-screening assay was employed the ability of candidates to inhibit the binding of GST-caspase 8 to His-caspase 8 was assessed; those that did were to be eliminated. In this counter-screening assay, the materials in each well were the same as in the primary screen, with the exception that GST-caspase 8 replaced GST-E6. Compounds 1 and 2 were able to inhibit the caspase 8/caspase 8 interaction about as well as they inhibited the E6/caspase 8 interaction, indicating that their actions were non-specific. However, spinacine (5) did not significantly inhibit the binding of caspase 8 to itself, providing evidence of its specificity for E6 (FIG. 2C). These results are summarized in Table 1, which shows that of the compounds tested, spinacine displayed the lowest EC₅₀ value for the inhibition of E6/caspase 8 and E6/E6AP interactions, while not inhibiting caspase 8/caspase 8 binding.

TABLE 1
EC₅₀ (μM) values of tested small molecules for
the indicated protein-protein interactions

| | | | | | Spinacine |
|---|---|---|---|---|---|
| | (1) | 2 | (3) | (4) | (5) |
| | [μM] | [μM] | [μM] | [μM] | [μM] |
| GST-E6/ | 0.63 | 1.038 | No Inhibition | No inhibition | 0.017 |
| His-caspase 8 | | | | | |
| GST-E6/ | 0.67 | 1.481 | No Inhibition | No inhibition | 0.02 |
| His-E6AP | | | | | |
| GST-caspase 8/ | 0.83 | 1.051 | No Inhibition | No inhibition | No |
| His-caspase 8 | | | | | Inhibition |

### Myricetin and Spinacine Sensitize SiHa Cells to TRAIL-Induced Apoptosis

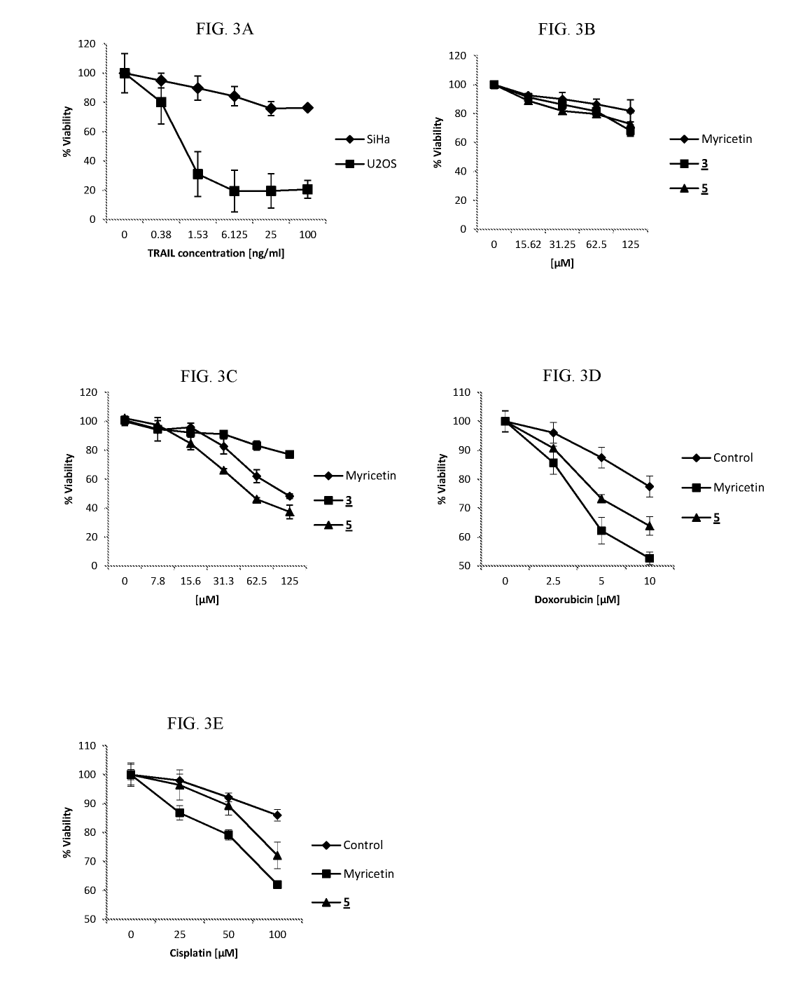

The molecules were then tested to determine whether those that block E6/caspase 8 binding in vitro could also act in the context of a cell. SiHa cells are an HPV⁺ cell line, derived from a cervical carcinoma, which serves as a commonly used model for HPV-associated malignancies. To determine whether HPV⁺ SiHa cells are resistant to TRAIL-induced apoptosis, SiHa cells were treated with TRAIL and cell viability was assessed. TRAIL-sensitive, HPV⁻ U2OS cells served as a positive control. The results (FIG. 3A) demonstrate that in comparison to U2OS cells, SiHa cells are relatively resistant to treatment with TRAIL, as predicted. Furthermore, both myricetin and spinacine displayed low toxicity to SiHa cells in the absence of TRAIL (FIG. 3B). Myricetin and/or spinacine were tested to determine if they could sensitize these HPV⁺ cells to TRAIL-induced apoptosis. The indicated concentrations (0 μM to 125 μM) of myricetin, (3) (negative control) and spinacine (5) were added to SiHa cells in the presence of TRAIL, and the data of FIG. 3C shows that in the presence of TRAIL, myricetin and spinacine were both able to reduce the viability of SiHa cells to 30-40% at a dose of 125 μM, in contrast to the negative control, 3. Table 2 lists and compares the concentrations of the three tested compounds required to induce death in 50% of SiHa cells in the presence or absence of TRAIL. The data demonstrates that both myricetin and spinacine induced significantly more cell death (an increase of 3.5 fold for myricetin and 8.6 fold for spinacine) in the presence than in the absence of TRAIL (Table 2). These results provide strong evidence that inhibiting the E6/caspase 8 interaction can restore the sensitivity of HPV⁺ cells to apoptotic signals.

TABLE 2
EC₅₀ (μM) values of the indicated compounds
for cell toxicity and for TRAIL-induced apoptosis

| | Myricetin | (3) | Spinacine (5) |
|---|---|---|---|
| | [μM] | [μM] | [μM] |
| Cell toxicity | 539.1 | 284.9 | 268.5 |
| TRAIL-induced apoptosis | 154.8 | No sensitization | 31.3 |

### Myricetin and Spinacine Increase the Sensitivity of SiHa Cells to Doxorubicin and Cisplatin

Both myricetin and spinacine can inhibit the binding of E6 to E6AP, an E3 ligase involved in the degradation of p53, in vitro. Based on this observation, myricetin and spinacine were tested to determine if they would increase the sensitivity of SiHa cells to two drugs, doxorubicin and cisplatin, that are thought to act by inducing intrinsic apoptosis through the activation of pathways that involve p53. It was found that as compared to the negative control, both myricetin and spinacine increased the sensitivity of SiHa cells to doxorubicin and cisplatin (FIGS. 3D and 3E). These results demonstrate that the ability of both myricetin and spinacine to inhibit the interaction between E6 and E6AP, resulting in a predicted increase in p53, results in the sensitization of HPV⁺ cells to inducers of p53-mediated apoptosis.

### Myricetin and Spinacine Increase Caspase 3/7 Activity

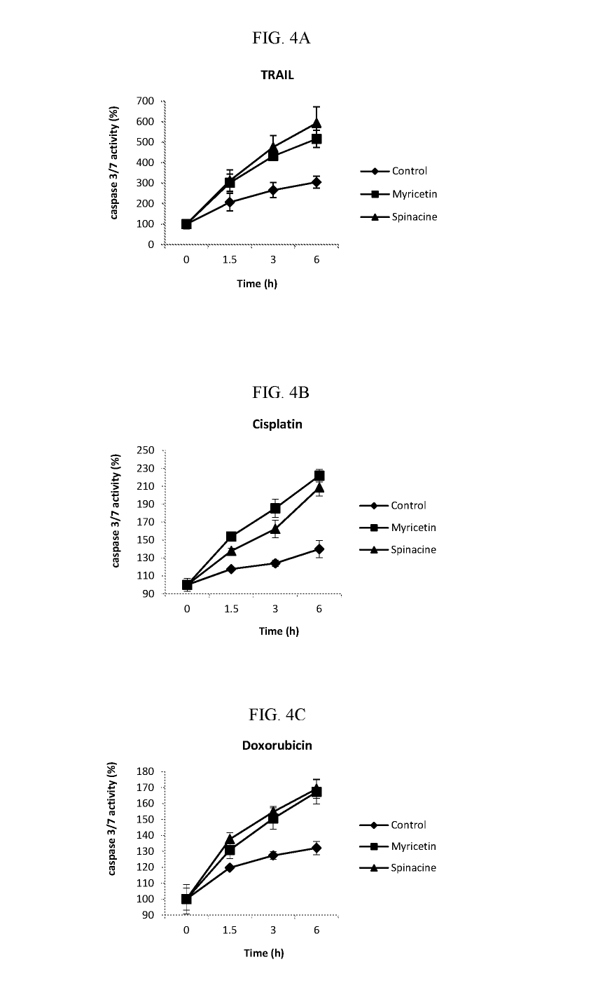

Activation of caspase 3/7 is an essential marker for both extrinsic and intrinsic apoptosis, and can be used to determine whether cell death occurs through the apoptotic pathway. The data of FIG. 2 demonstrates that both myricetin and spinacine can inhibit the E6/caspase 8 and E6/E6AP interactions in vitro, resulting in increased cell death. If this cell death occurred through apoptosis, then pre-treatment of cells with either myricetin or spinacine should increase the level of TRAIL-, cisplatin- and doxorubicin-induced activation of caspase 3/7. To test this, caspase 3/7 activity was measured in SiHa cells following TRAIL, cisplatin and doxorubicin treatment in the presence or absence of myricetin or spinacine. As shown in FIG. 4A, it was found that the caspase 3/7 activity increased significantly when myricetin or spinacine were combined with TRAIL as compared to controls. Similar results were obtained following treatment with cisplatin and doxorubicin (FIGS. 4B, 4C). These data demonstrate that the increased cell death induced by the combination of apoptotic inducers and myricetin/spinacine occurs through the apoptotic pathway.

### Myricetin and Spinacine Re-sensitize HPV⁺ Cells to TRAIL-Induced Apoptosis by Blocking the Binding of HPV E6 to Caspase 8

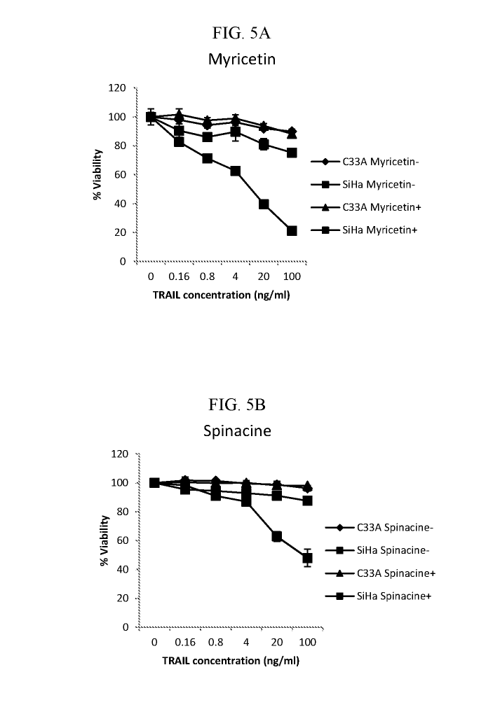

To determine whether myricetin and spinacine re-sensitize SiHa cells to TRAIL-induced apoptosis by specifically blocking the binding of E6 to caspase 8, responses in the HPV⁺ SiHa cells were compared with those from the HPV⁻ human cervical carcinoma cell, C33A, as these cells do not express E6. It was found that although the HPV⁺ SiHa cells displayed resistance to TRAIL at concentrations up through 100 ng/ml, their sensitivity increased dramatically in the presence of 100 μM myricetin. In contrast, the HPV⁻ C33A cells remained resistant to TRAIL-mediated apoptosis even in the presence of myricetin (FIG. 5A). Similar results were also found following pre-treatment with 50 μM spinacine (FIG. 5B). Together, these results demonstrate that myricetin and spinacine can re-sensitize HPV⁺ cells to TRAIL-mediated apoptosis by specifically blocking the binding of HPV E6 to apoptotic proteins such as caspase 8.

### Myricetin and Spinacine Increase the Levels of Caspase 8 and p53 in SiHa Cells

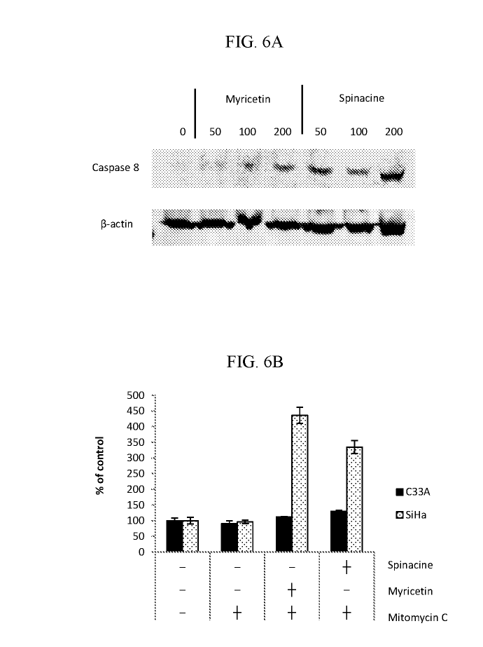

The data described above demonstrates that myricetin and spinacine are able to increase caspase 3/7 activity (FIG. 4) and to sensitize SiHa cells to TRAIL- (FIGS. 3A-C and 5) and p53- (FIGS. 3D-E) induced apoptosis. If this ability is due to inhibition of E6 functions, then an increase in the levels of caspase 8 and p53 should be observable in SiHa cells following treatment with myricetin and spinacine. To test this prediction, SiHa cells were treated with 50, 100, and 200 μM of myricetin or spinacine for 24 hr, and then measured the level of caspase 8 by immunoblot. The results (FIG. 6A) demonstrate that treatment with myricetin caused an increase in caspase 8 levels at doses of 100 μM and 200 μM, while spinacine caused an increase in caspase 8 at doses from 50 to 200 μM. To ask how p53 levels were affected, an ELISA assay was employed. For comparison, C33A cells, which lack HPV E6 and express mutant p53, were also treated with the same concentrations of spinacine and myricetin. Mitomycin C was included in the assay to cause DNA damage and therefore increase p53 levels. The results from this experiment (FIG. 6B) demonstrate that treatment of SiHa cells with myricetin caused a 4.4-fold increase in p53 at a dose of 200 μM, and that treatment with spinacine caused 3.4-fold increase in p53 at a dose of 100 μM, as compared to untreated cells. Furthermore, there was no significant change in p53 levels in C33A cells, confirming the conclusion that myricetin and spinacine impact apoptotic pathways only in the presence of E6. Together, these results demonstrate that these small molecules are able to increase the sensitivity of SiHa cells to both TRAIL- and p53-induced apoptosis by blocking the ability of E6 to bind to its cellular partners, thereby reducing the degradation of caspase 8 and p53 in HPV⁺ SiHa cells

### Myricetin and Spinacine Sensitize HPV⁺ Head and Neck Squamous Cell Carcinoma (HNSCC) Cells to TRAIL-induced Cell Death

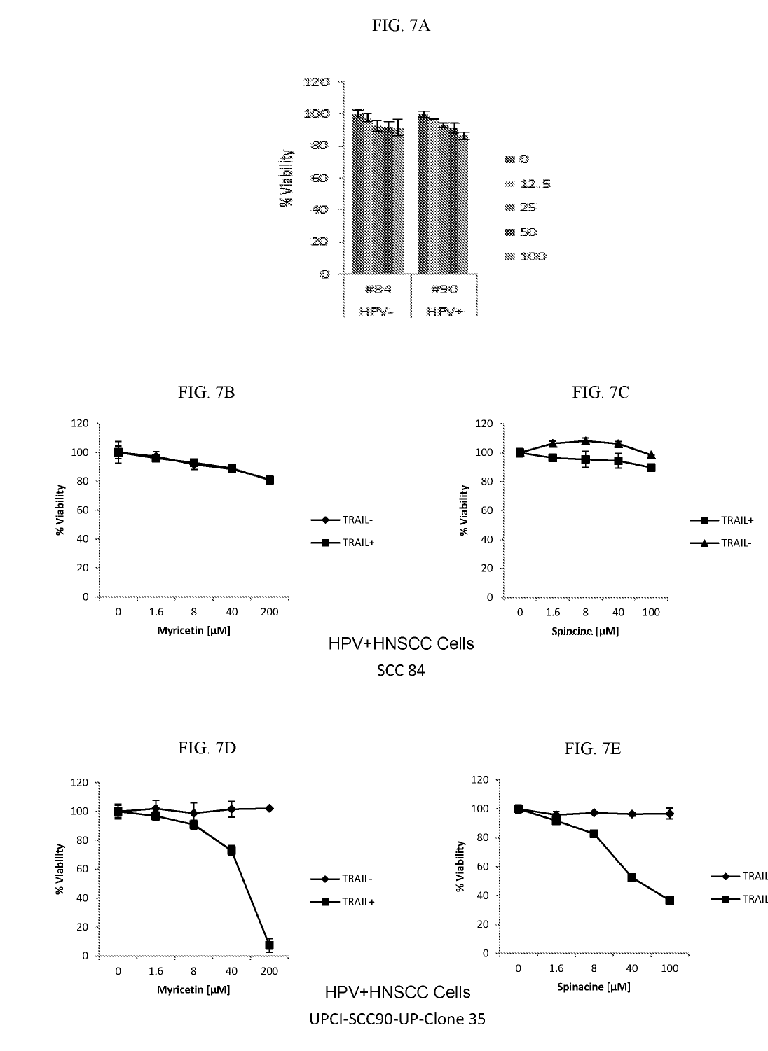

As shown above, myricetin and spinacine sensitize SiHa cells to TRAIL and chemotherapy drugs. These data provide proof-of-principle that small molecule inhibitors can block HPV E6 functions and trigger HPV⁺ cervical cancer cell lines to undergo apoptosis or other cell death inducing processes. Other types of HPV⁺ cancer cells were examined, and in particular, head and neck squamous cell carcinoma (HNSCC) cells can also be sensitized in this manner. All selected cell lines demonstrated resistance to TRAIL at concentrations as high as 100 ng/ml (FIG. 7A). For the combination experiments, 50 ng/ml TRAIL was applied in combination with either myricetin (0-200 μM) or spinacine (0-100 μM). As shown in FIG. 7B, the HPV⁻ HNSCC cell line (SCC84, #84) was resistant to TRAIL in both the presence and absence of myricetin or spinacine. In contrast (FIG. 7C), TRAIL-induced cell death was enhanced 30% in UPCI-SCC90-UP-Clone 35, #90. Furthermore, 40 μM spinacine was able to enhance TRAIL-induced cell death in the HPV⁺ #90 cell lines up to 50%. Together, these results indicate that both myricetin and spinacine sensitize HPV⁺, but not HPV⁻, HNSCC cell lines to TRAIL-induced apoptosis, confirming previous results that these small molecules block HPV E6 functions.

### Myricetin and Spinacine Sensitize HPV⁺ HNSCC Cells to Doxorubicin

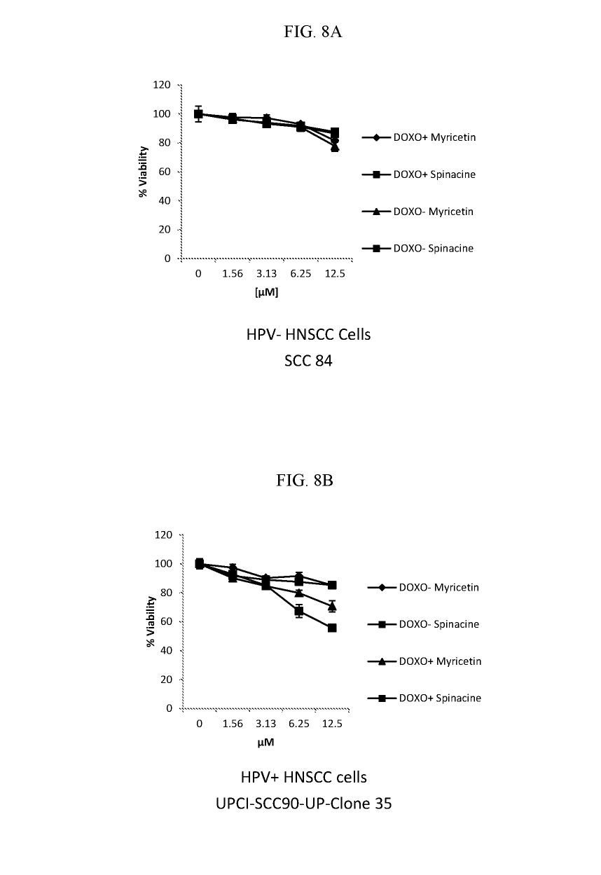

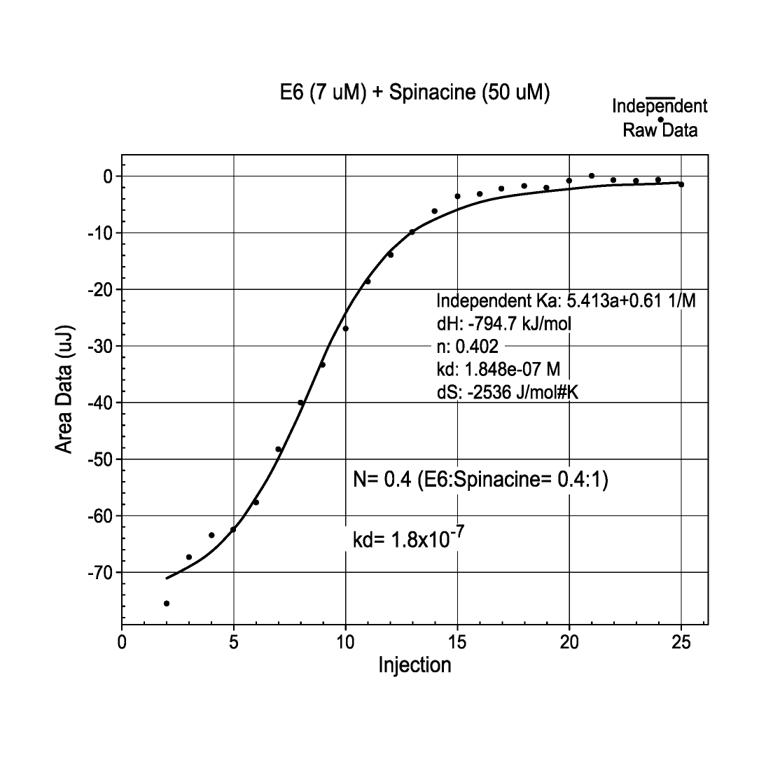

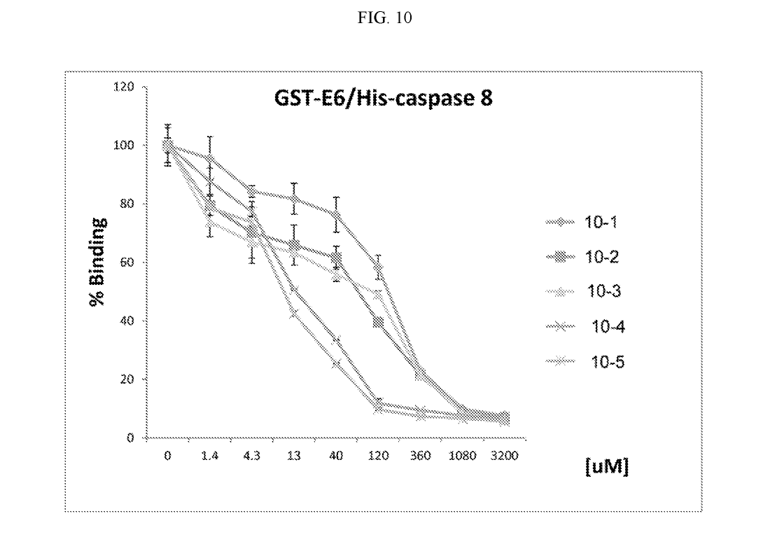

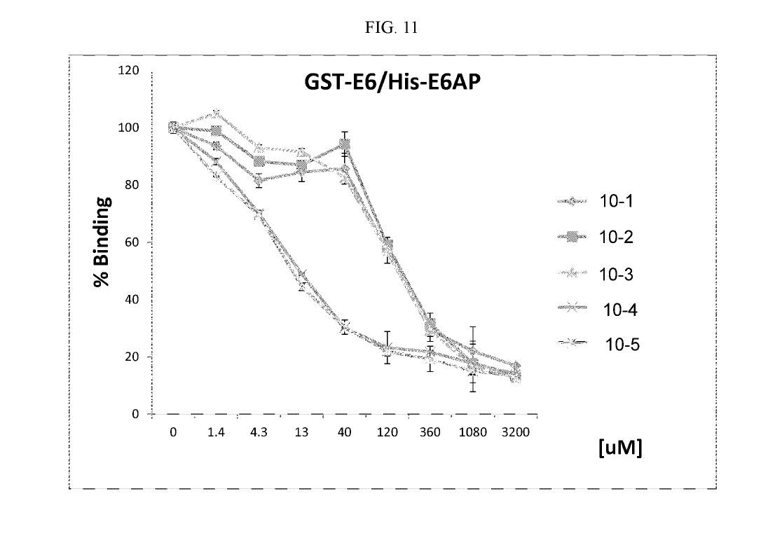

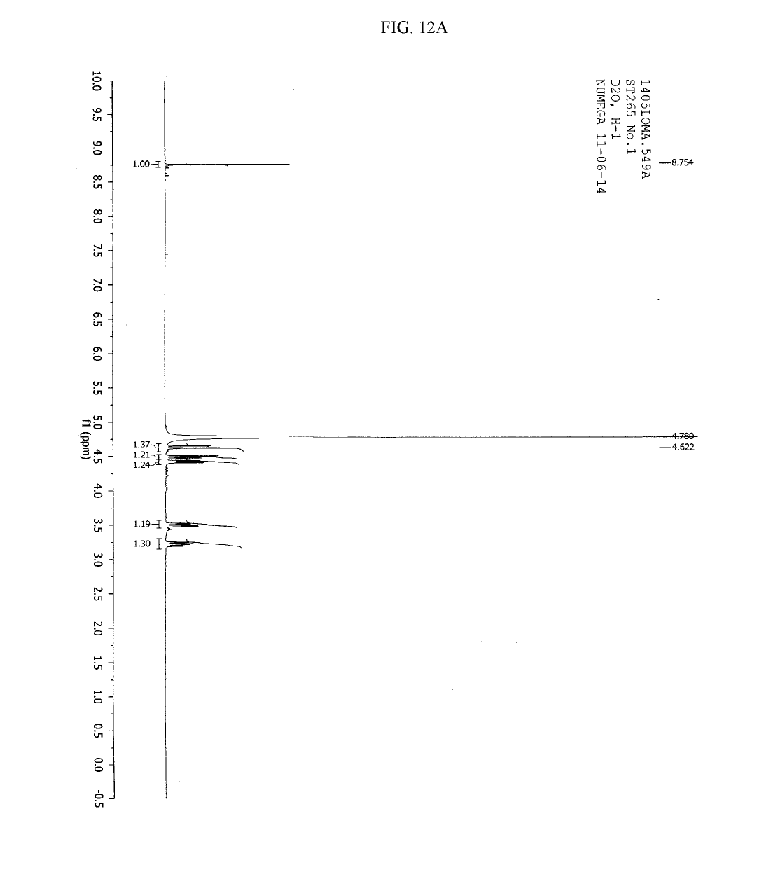

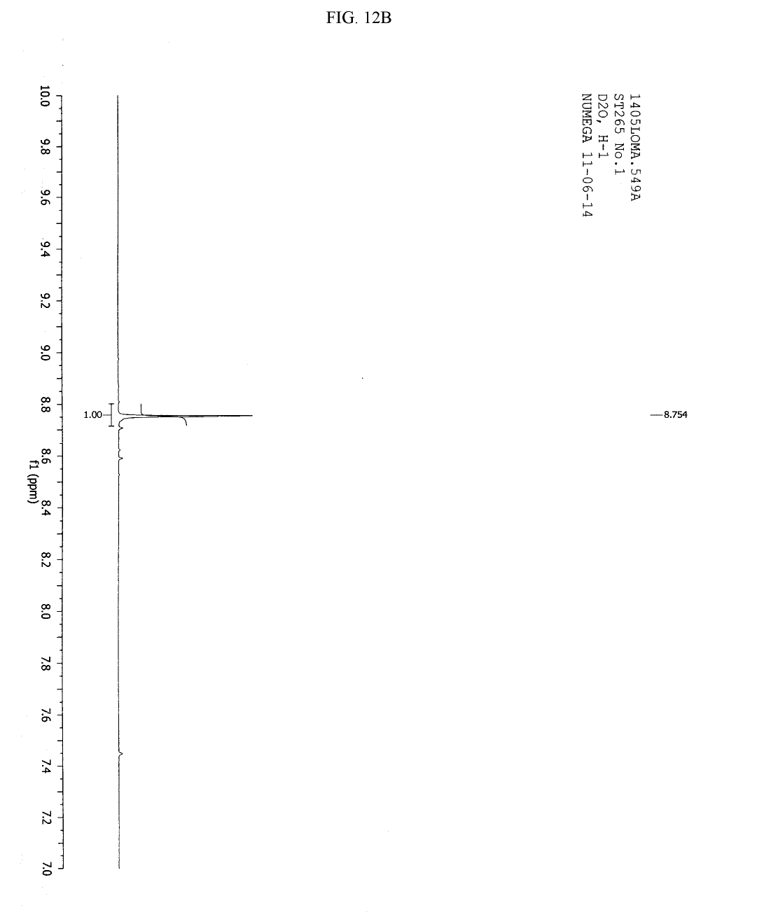

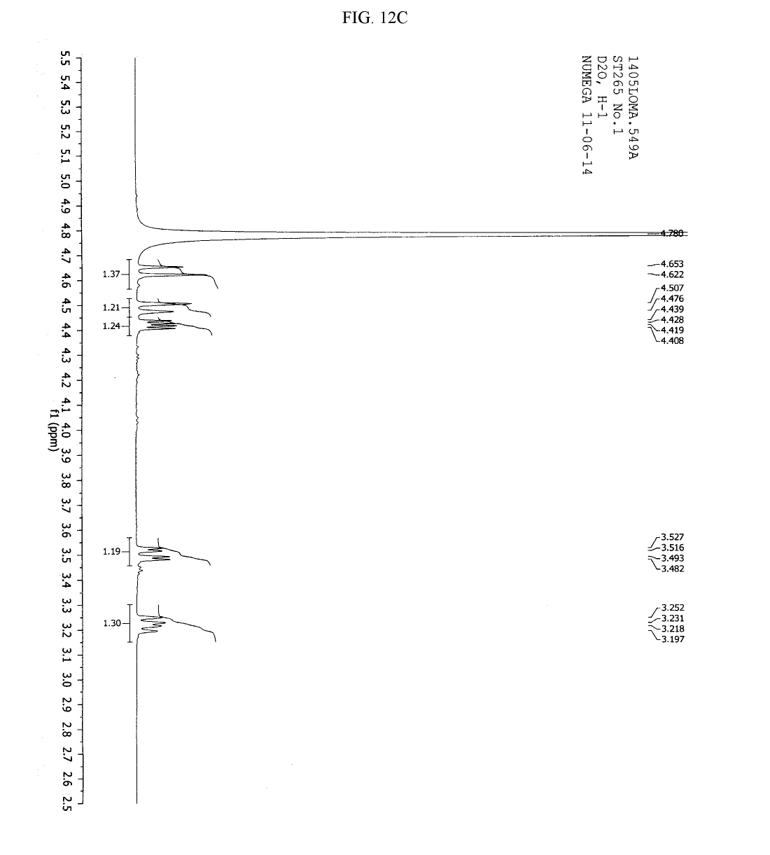

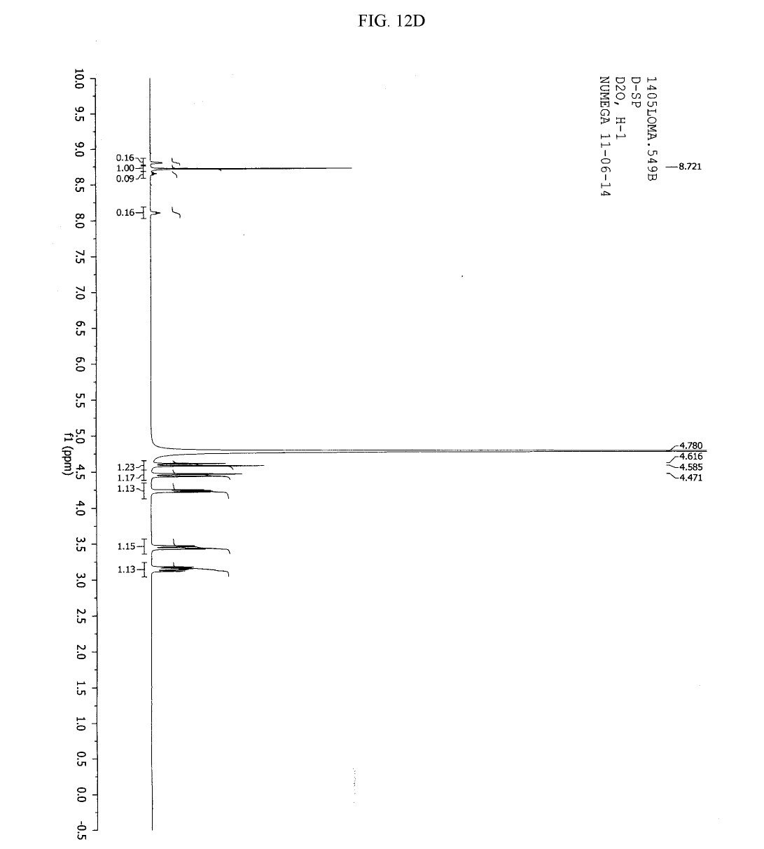

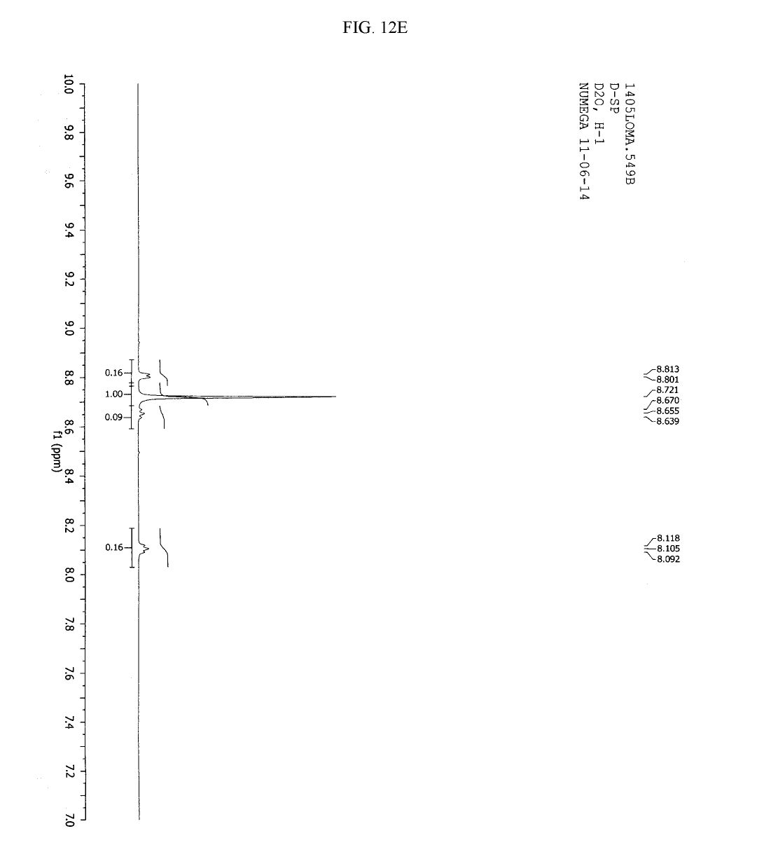

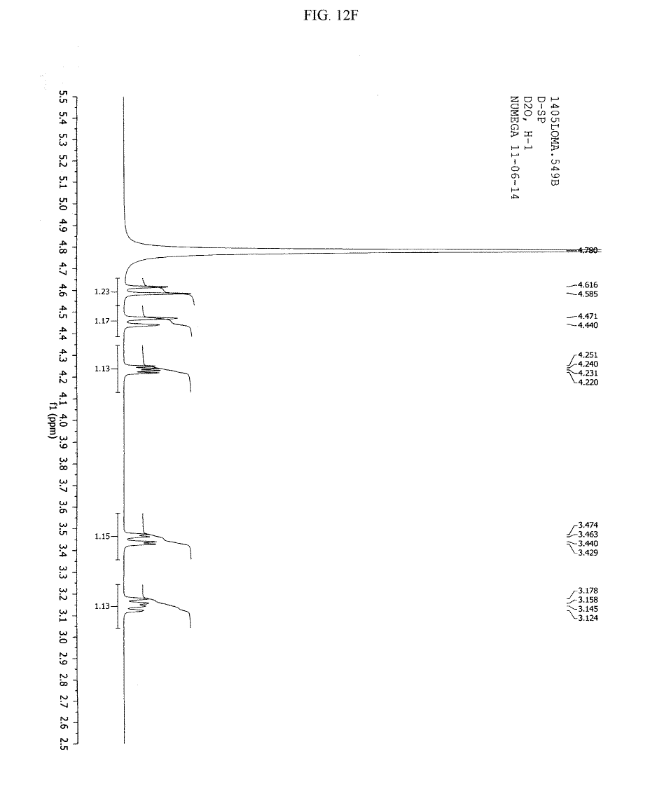

The results described above indicate that myricetin and spinacine sensitize HPV⁺ HNSCC cells to TRAIL-induced apoptosis. To determine whether this sensitization also occurs in the context of chemotherapeutic agents, several HNSCC cell lines were tested for their responses to combination treatments. As shown in FIG. 8A, the HPV⁻ HNSCC cell line SCC 84 (#84) was resistant to doxorubicin in both the presence and the absence of myricetin and spinacine. However, HPV⁺ HNSCC cancer cell lines, such as UPCI-SCC90-UP-Clone 35 (#90), displayed an increase in cell death when treated by combinations of 2 μM doxorubicin plus the indicated small molecules (FIG. 8B). In these experiments, spinacine demonstrated a higher efficiency than did myricetin, sensitizing #90 to doxorubicin by up to 50% at a concentration of 12.5 μM. Together, these data demonstrate that myricetin and spinacine can sensitize both cervical and oral HPV⁺ cancer cells to chemotherapeutic agents such as doxorubicin.

### Targeting of HPV Proteins

Small molecules targeting any of the HPV proteins and available for clinical use are desirable, as these sorts of small, inhibitory molecules can be developed to expand and enhance the limited therapeutic options currently available for HPV-associated malignancies (D'Abramo, C. M. and J. Archambault, Small molecule inhibitors of human papillomavirus protein-protein interactions. The open virology journal, 2011. 5: p. 80-95; Phelps, W. C., J. A. Barnes, and D. C. Lobe, Molecular targets for human papillomaviruses: prospects for antiviral therapy. Antiviral chemistry & chemotherapy, 1998. 9(5): p. 359-77). E6 and E7 oncogenes are expressed at relatively high levels during cancer development and therefore have the potential to serve as useful targets. For example, inhibition of E6 is predicted to lead to increased cell death, as E6 normally functions to block both intrinsic and extrinsic apoptotic pathways (Hebner, C., M. Beglin, and L. A. Laimins, Human papillomavirus E6 proteins mediate resistance to interferon-induced growth arrest through inhibition of p53 acetylation. Journal of virology, 2007. 81(23): p. 12740-7). One approach to blocking the interactions between E6 and its cellular partners is to use peptides, and peptide inhibitors have been identified that can specifically inhibit the interactions between E6 and caspase 8 and FADD (Tungteakkhun, S. S., et al., The interaction between human papillomavirus type 16 and FADD is mediated by a novel E6 binding domain. Journal of virology, 2008. 82(19): p. 9600-14) or between E6 and E6AP (Tungteakkhun, S. S., et al., The interaction between human papillomavirus type 16 and FADD is mediated by a novel E6 binding domain. Journal of virology, 2008. 82(19): p. 9600-14; 34. Liu, Y., et al., Design and characterization of helical peptides that inhibit the E6 protein of papillomavirus. Biochemistry, 2004. 43(23): p. 7421-31). As compared with peptide inhibitors, however, small molecules such as those described herein possess numerous advantages because they are more stable, penetrate target cells more easily, and can more readily be modified and optimized by organic chemists during drug development. Some progress has been made in this area, as published studies have identified several compounds (some with EC50 values between 17 and 29 μM) that could inhibit the interaction between E6 and E6AP (D'Abramo, C. M. and J. Archambault, Small molecule inhibitors of human papillomavirus protein-protein interactions. The open virology journal, 2011. 5: p. 80-95; Baleja, J. D., et al., Identification of inhibitors to papillomavirus type 16 E6 protein based on three-dimensional structures of interacting proteins. Antiviral research, 2006. 72(1): p. 49-59). However, no structure-activity relationship (SAR) has been carried out for those compounds because of the limited data set, and none of these findings have yet led to clinically useful interventions. While myricetin has been reported to inhibit interactions between E6 and caspase 8 in vitro, and preliminary results indicate that myricetin binds to E6 at a ratio of 2:1, unfortunately, myricetin is known to inhibit a number of cellular proteins (including several kinases), indicating a lack of sufficient specificity. In addition, the high EC50 value observed in the context of a cell-based model system suggests that its ability to negatively affect tumors may be limited, perhaps because its polar nature limits its ability to enter cells. As polar groups are an important feature in assessing SARs, identification of targets in this series of compounds would be difficult without resorting to a pro-drug approach. Therefore, additional screening was conducted with libraries containing more ‘lead-like’ members that are expected to provide better platforms for commercialization.

Of the five tested compounds, spinacine (5) was best able to specifically inhibit the interactions of E6 with both caspase 8 and E6AP demonstrating the lowest EC50 values for both E6/Caspase 8 and E6/E6AP binding. However, it did not inhibit caspase 8/caspase 8 binding, thus demonstrating the desired specificity. In the experiments that involved treating SiHa cells with TRAIL, the EC50 values demonstrate that, as compared with myricetin, spinacine may be better able to penetrate into cells and thus re-sensitize cells more efficiently to TRAIL-induced apoptosis (Table 2).

Therapies based on TRAIL-mediated apoptosis, alone or in combination with other agents, are attractive possibilities for the treatment of many different types of cancer (Bellail, A. C., et al., TRAIL agonists on clinical trials for cancer therapy: the promises and the challenges. Rev Recent Clin Trials, 2009. 4(1): p. 34-41; El-Zawahry, A., J. McKillop, and C. Voelkel-Johnson, Doxorubicin increases the effectiveness of Apo2L/TRAIL for tumor growth inhibition of prostate cancer xenografts. BMC Cancer, 2005. 5: p. 2; Mom, C. H., et al., Mapatumumab, a fully human agonistic monoclonal antibody that targets TRAIL-R1, in combination with gemcitabine and cisplatin: a phase I study. Clin Cancer Res, 2009. 15(17): p. 5584-90; Naka, T., et al., Effects of tumor necrosis factor-related apoptosis-inducing ligand alone and in combination with chemotherapeutic agents on patients' colon tumors grown in SCID mice. Cancer Res, 2002. 62(20): p. 5800-6). However, these treatments are unlikely to be helpful in the context of HPV on their own, owing to the ability of E6 to compromise apoptotic pathways. It was found that the combination of TRAIL with small molecules can re-sensitize E6-expressing cells to TRAIL-induced apoptosis, potentially filling this gap. In addition to sensitizing cells to TRAIL, it was found that both myricetin and spinacine sensitized cells to intrinsic apoptosis triggered by chemotherapy drugs, such as doxorubicin and cisplatin. This may be clinically relevant, because cervical cancer tends to be relatively resistant to chemotherapeutic treatments, such as cisplatin (Rein, D. T. and C. M. Kurbacher, The role of chemotherapy in invasive cancer of the cervix uteri: current standards and future prospects. Anticancer Drugs, 2001. 12(10): p. 787-95). Myricetin required a higher concentration (100 μM) than did spinacine (50 μM) to inhibit binding and to sensitize cells, suggesting that spinacine is a more efficient agent. Taken together, these data indicate that myricetin and spinacine can block the binding of E6 to multiple apoptotic proteins, including caspase 8 and E6AP/p53, thereby reactivating the E6-compromised apoptotic pathways and rendering HPV+ cells sensitive to both intrinsic and extrinsic inducers of apoptosis. The ability of myricetin and spinacine to sensitize HPV+ SiHa cells to apoptosis induced by TRAIL and chemotherapy agents suggests the same effects could be seen in HPV+ HNC cells. It was found that the result from HNC cells closely mirrored those from cervical cells, indicating the methods and compositions presented herein should have broad applicability to HPV-associated malignancies regardless of the site of origin. The data suggest a mode of action in which the small molecule interacts directly with E6, either destabilizing the virus protein and changing its conformation, or blocking the interactions by direct interference. If the latter, one possibility is that myricetin/spinacine binds to a region on E6 required by both E6AP and caspase 8. These results regarding the combination of small molecules with TRAIL/chemotherapeutic agents can be employed in therapeutic approaches for the effective and selective killing of HPV+ cancer cells and for developing therapeutic strategies to treat HPV-mediated cancers.

### Experiments

### Protein Purification

The construction of the pGEX-E6, pTriEx-E6AP, and pTriEx-Caspase-8 DED plasmids has been reported (Tungteakkhun, S. S., et al., The interaction between human papillomavirus type 16 and FADD is mediated by a novel E6 binding domain. Journal of virology, 2008. 82(19): p. 9600-14). Expression and purification of GSTE6, His-E6AP, and His-Caspase-8 DED were carried out as previously described (Tungteakkhun, S. S., et al., The interaction between human papillomavirus type 16 and FADD is mediated by a novel E6 binding domain. Journal of virology, 2008. 82(19): p. 9600-14; Tungteakkhun, S. S., et al., The full-length isoform of human papillomavirus 16 E6 and its splice variant E6* bind to different sites on the procaspase 8 death effector domain. Journal of virology, 2010. 84(3): p. 1453-63). GST-tagged and His-tagged proteins were diluted into GST dilution buffer (PBS pH 8.0, 5% glycerol, 2 mM DTT) and His dilution buffer (20 mM Hepes pH 7.4, 150 mM NaCl, 2 mM KCl, 5% glycerol, 2 mM DTT), respectively. Protein concentration was measured using Coomassie Plus—The Better Bradford Assay Reagent (Thermo Scientific). Purity of the isolated proteins was estimated following separation by SDS-PAGE and Coomassie staining.

### Small Molecule Library and Acquisition of Additional Compounds

The 2,000-compound small molecule library (ActiProbe 2K) was acquired from TimTec, LLC (Newark, Del., USA) and was chosen because it encompasses a highly diverse selection of lead-like compounds. Five additional derivatives were purchased from Sigma (St. Louis, Mo., USA) (benzimidazole, 2-(methoxymethyl)-1H-benzimidazole, and 3-(1H-Indol-1-yl)propan-1-amine methanesulfonate) and TimTec (1H-benzimidazole-1-methanol and 4,5,6,7-tetrahydro-1H-imidazo[4,5-c]pyridine-6-carboxylic acid).

### Screening of the Small Molecular Library

Alpha-Screen™ technology was used to assess the interactions between GST-E6, GST-caspase 8, His-E6AP, and His-caspase 8. Binding assays were performed in white 384-well plates (Perkin-Elmer) in a total volume of 25 μl as previously described (Tungteakkhun, S. S., et al., The interaction between human papillomavirus type 16 and FADD is mediated by a novel E6 binding domain. Journal of virology, 2008. 82(19): p. 9600-14). Briefly, 5 μl (50 ng) of GST-E6 and 5 μl (87.5 ng) of His-caspase 8 were included in each reaction mixture with 5 μl blocking buffer (0.5 mg BSA, 0.5% Tween 20 in PBS) in the absence or presence of 10 μM of each test chemical. Members of the library were present at M in DMSO. After a one-hour incubation of the mixture at room temperature, 5 μl donor beads and 5 μl acceptor beads (Perkin-Elmer) were added to each well according to the manufacturer's protocol. The mixture was incubated in the dark at room temperature overnight, and the emitted signal was detected using the Envision Multilabel plate reader (Perkin-Elmer). In the presence of test chemicals, the binding affinity was calculated as a percentage of the binding in the presence of carrier only (DMSO).

### Cell Culture

U2OS, SiHa and C33A cells were obtained from the America Type Culture Collection (Manassas, Va., USA) and cultured in Eagle minimal essential medium (Invitrogen, Carlsbad, Calif., USA) supplemented as described previously (Tungteakkhun, S. S., et al., The interaction between human papillomavirus type 16 and FADD is mediated by a novel E6 binding domain. Journal of virology, 2008. 82(19): p. 9600-14). HNSCC cell lines were obtained from several sources: UD-SCC2-TC-Clone 5(#2TC), UPCI-SCC90-UP-Clone 35 (#90), and SCC 84 were a gift from Dr. John Lee, Sanford Research (South Dakota, S.D., USA). HNSCC cells were cultured in Dulbecco's Modified Eagle Medium (Mediatech, VA) supplemented with 10% of FBS.

### Cell Viability Assay

The extracellular domain of human TRAIL was cloned into a pTriEx expression plasmid containing N-terminal Hisx6 tag. His-TRAIL was expressed in the E. coli BL-21 pLys strain and purified as previously described. Doxorubicin and cisplatin were purchased from Sigma. His-TRAIL, doxorubicin, and cisplatin were diluted in PBS to the desired concentration before using. To measure cell survival following treatment with TRAIL, cisplatin and doxorubicin, SiHa (2×10⁴/well), C33A (1×10⁴/well), and HNSCC cells (2×10⁴/well) were seeded into 96-well plates and allowed to adhere overnight. Small molecules at the desired concentration were added and incubated at 37° C. for 4 h. As indicated, TRAIL, cisplatin or doxorubicin was then added. The TRAIL-treatment group contained cycloheximide (5 μg/ml) to inhibit de novo protein synthesis, and the cells were incubated for 16 h prior to measuring cell viability by the MTT assay preformed as described previously (Filippova, M., L. Parkhurst, and P. J. Duerksen-Hughes, The human papillomavirus 16 E6 protein binds to Fas-associated death domain and protects cells from Fas-triggered apoptosis. The Journal of biological chemistry, 2004. 279(24): p. 25729-44). All experiments were repeated at least three times (three biological replicates, carried out on different days), with each experimental group measured in triplicate within each of these individual experiments. Data presented are from a representative experiment.

### Caspase Activity Assay

Cells were plated into 96-well plates at a density of 2×10⁴ cells per well and incubated overnight. Small molecules were then added and incubated at 37° C. for 4 h. TRAIL (100 ng/ml), cisplatin (50 μM), and doxorubicin (2 μM) were then added. Cycloheximide (5 μg/ml) was added along with TRAIL at the indicated time points. Caspase 3/7 activity was measured using the fluorogenic substrate CellTitier-Glo for caspase 3/7 activity (Promega, Fitchburg, Wis., USA) following the manufacturer's instructions. Briefly, cells were lysed by the addition of 20 μl of 5× passive lysis buffer (Promega). The plate was put on an orbital shaker and incubated for 10 min at room temperature. 20 μl of cell lysates were transferred to white plates, and either substrate alone or substrate plus the caspase 3/7 inhibitor was added to the appropriate wells. After a 10 min incubation, the released fluorophore was measured using a plate-reading fluorimeter (Flx800, Bio-Tek Instrument Co., Vinooski, Vt., USA). The activity in wells treated with inhibitor was subtracted from the activity in wells lacking inhibitor. The resulting difference was expressed as a percentage of the caspase activity of the untreated cells.

### Immunoblot Analysis

Cells (1×10⁶) were collected and washed with PBS. All liquid was removed by centrifugation at 3,000 rpm for 5 min at 4° C. The cell pellet was lysed in 100 l lysis buffer (50 mM Tris-HCl, pH 7.4, 150 mM NaCl, 1% NP-40, 1 mM EDTA, 5% glycerol, 1 mM dithiothreitol, 1 mM phenylmethylsulfonyl fluoride, with one tablet of protease inhibitor mixture (Roche Molecular Biochemicals, Basel, Switzerland) per 10 ml of buffer added just prior to use) for 10 min on ice. Lysates (10 to 40 μg total protein/lane) were then subjected to 10% or 12% sodium dodecyl sulfate-polyacrylamide gel electrophoresis (SDS-PAGE) and transferred to PVDF membranes using the iBlot® dry blotting system (Invitrogen). Anti-caspase 8 monoclonal antibodies (BD Pharmingen, Franklin, N.J., USA) and anti-β-actin monoclonal antibodies (Sigma) were applied at 1:5000 dilutions. The secondary antibodies used were goat anti-mouse IRDye800 (LI-COR Biosciences) for caspase 8 and goat anti-mouse Dy680 (LI-COR Biosciences, Lincoln, Nebr., USA) for β-actin at 1:30,000 dilutions. Signals were measured using the Odyssey Infrared Imaging system (LI-COR Biosciences) and expressed in relative light units.

### p53 ELISA

The p53 ELISA was performed as described previously (Filippova, M., et al., The large and small isoforms of human papillomavirus type 16 E6 bind to and differentially affect procaspase 8 stability and activity. J Virol, 2007. 81(8): p. 4116-29), with some modifications. Antibodies secreted by clone pAb122 (hybridoma obtained from the ATCC, antibodies purified from the culture medium using protein-A Sepharose) were used as monoclonal capture antibodies. This antibody has a broad specificity, binding to both human and mouse forms of p53 and to both wild-type and mutant forms of the protein. These antibodies were absorbed (50 μL per well, 4 μg/mL) onto the surfaces of a Nunc-immuno plate, MaxiSorp Surface (NalgeNunc International, Rochester, N.Y., USA) by incubation overnight at 4° C. The plates were then washed on an Auto Strip Washer, ELx50 (Bio-Tek Instruments, Inc.) using PBST (PBS plus 0.1% Tween 20) six times. Nonspecific binding sites were blocked by incubation with 200 μL per well of PBS including 10% calf serum (Invitrogen) (blocking buffer) for 2 h at room temperature, followed by washing as described above. 100 μL of each cell lysate to be tested were then added to the coated wells and allowed to incubate overnight at 4° C. After washing, 100 μL of a solution containing 4 g/mL biotinylated anti-p53 antibodies (polyclonal, produced in sheep, Roche) diluted into blocking buffer was added to each well and allowed to incubate for 45 min at room temperature. The plates were washed, and then the avidin-peroxidase conjugate (Sigma; 100 μL/well, 2.5 μg/mL diluted into blocking buffer) was added and allowed to incubate for 30 min at room temperature. After washing, 100 μL of the substrate (0.3 mg/mL ABTS (2,2′-azino-di-(3-ethylbenzthiazolin sulfonate) dissolved into 0.1 M citric acid, pH 4.35, with 1 μL/mL 30% H₂O₂ added just before use) was added to each well and allowed to incubate for approximately 30 min. The absorbance at 405 nm was read with a microplate reader (Dynex Technologies; MRX Revelation software, Chantilly, Va., USA). The protein concentration of each lysate was also measured using the BCA method (Pierce, Chantilly, Va., USA) and used to normalize the measured p53 values for possible variations in the number of cells per well. Each p53 value (obtained from the ELISA assay) was divided by the protein concentration to obtain a normalized p53 value (ng p53 per mg total protein). The average and standard deviation of the replicates (a minimum of three) were calculated, normalized to the control, and used to prepare the graph.

### Methods of Treatment

It is generally preferred to administer the compounds of various embodiments in an intravenous or subcutaneous unit dosage form of a pharmaceutical composition; however, other routes of administration are also contemplated. Contemplated routes of administration include but are not limited to oral, parenteral, intravenous, transcutaneous, transmembranal, and subcutaneous. The compounds of various embodiments can be formulated into liquid preparations for, e.g., oral administration. Suitable forms include suspensions, syrups, elixirs, and the like. Particularly preferred unit dosage forms for oral administration include tablets and capsules. A suitable form for trasmembranal administration to, e.g., cervical tissue, includes a douche. Unit dosage forms configured for administration once a day are particularly preferred; however, in certain embodiments it can be desirable to configure the unit dosage form for administration twice a day, or more.

The pharmaceutical compositions of various embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. In certain embodiments it can be desirable to maintain the active compound in the reduced state. Accordingly, it can be desirable to include a reducing agent, such as vitamin C, vitamin E, or other reducing agents as are known in the pharmaceutical arts, in the formulation.

Viscosity of the pharmaceutical compositions can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents.

A pharmaceutically acceptable preservative can be employed to increase the shelf life of the pharmaceutical compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected. Reducing agents, as described above, can be advantageously used to maintain good shelf life of the formulation.

The compounds of various embodiments can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the pharmaceutical compositions can be provided as a tablet, aqueous or oil suspension, dispersible powder or granule, emulsion, hard or soft capsule, syrup or elixir. Compositions intended for oral use can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Formulations for oral use can also be provided as hard gelatin capsules, wherein the active ingredient(s) are mixed with an inert solid diluent, such as calcium carbonate, calcium phosphate, or kaolin, or as soft gelatin capsules. In soft capsules, the active compounds can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the active ingredient in admixture with fillers such as lactose, binders such as starches, and/or lubricants such as talc or magnesium stearate and, optionally, stabilizers. In instances where it is desirable to maintain a compound of a various embodiment in a reduced form (in the case of certain active metabolites), it can be desirable to include a reducing agent in the capsule or other dosage form.

Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s), preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the active ingredients in admixture with non-toxic pharmaceutically acceptable excipients including inert materials. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of a compound of the various embodiments, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. In certain embodiments it can be preferred to incorporate two or more of the therapeutic agents to be administered into a single tablet or other dosage form (e.g., in a combination therapy); however, in other embodiments it can be preferred to provide the therapeutic agents in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragacanth, or alginic acid or salts thereof.

Binders can be used to form a hard tablet. Binders include materials from natural products such as acacia, tragacanth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, can be included in tablet formulations.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations can be employed wherein the amifostine or analog(s) thereof is incorporated into an inert matrix that permits release by either diffusion or leaching mechanisms. Slowly degenerating matrices can also be incorporated into the formulation. Other delivery systems can include timed release, delayed release, or sustained release delivery systems.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments can be added for identification or to characterize different combinations of active compound doses.

When administered orally in liquid form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

When a compound of the various embodiments is administered by intravenous, parenteral, or other injection, it is preferably in the form of a pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of the injection can be adjusted depending upon various factors, and can comprise a single injection administered over the course of a few seconds or less, to 0.5, 0.1, 0.25, 0.5, 0.75, 1, 2, 3, 4, 5, 6, 7, 8, 9, 10, 11, 12, 13, 14, 15, 16, 17, 18, 19, 20, 21, 22, 23, or 24 hours or more of continuous intravenous administration.

The compositions of the various embodiments can additionally employ adjunct components conventionally found in pharmaceutical compositions in their art-established fashion and at their art-established levels. Thus, for example, the compositions can contain additional compatible pharmaceutically active materials for combination therapy (such as supplementary antimicrobials, antipruritics, astringents, local anesthetics, anti-inflammatory agents, reducing agents, and the like), or can contain materials useful in physically formulating various dosage forms of the various embodiments, such as excipients, dyes, thickening agents, stabilizers, preservatives or antioxidants.

In certain embodiments, spinacine (4,5,6,7-Tetrahydro-1H-imidazo(4,5-c)pyridine-6-carboxylic acid) and/or DIPC (6,7-dihydroimidazo[5,4-c]pyridine-6-carboxylic acid) can be administered with one or more TNF superfamily ligands. Most TNF ligands are type II transmembrane proteins whose extracellular domains can be cleaved by specific metalloproteinases to generate soluble cytokines. Cleaved and non-cleaved ligands are active as non-covalent homotrimers except for Lymphotoxin beta, which forms heterotrimers with TNF-beta and BAFF, which forms heterotrimers with APRIL. TNF family ligands are characterized by a stalk of varying length connecting the transmembrane domain to the core region, which contains the hallmark structure of TNF family ligands, the TNF homology domain (THD). The THD is an anti-parallel beta-pleated sheet sandwich with a “jelly-roll” topology. Conserved residues within the beta-strands provide specific inter-subunit contacts, which stabilize the trimeric structure. Sequences in the loops connecting adjacent beta-strands are family member-specific and are important for conferring receptor specificity. TNF superfamily ligands include, but are not limited to, 4-1BB Ligand/TNFSF9; APRIL/TNFSF13; BAFF/BLyS/TNFSF13B; CD27 Ligand/TNFSF7; CD30 Ligand/TNFSF8; CD40 Ligand/TNFSF5; EDA/Ectodysplasin; EDA-A2/Ectodysplasin A2; Fas Ligand/TNFSF6; GITR Ligand/TNFSF18; LIGHT/TNFSF14; Lymphotoxin; Lymphotoxin beta/TNFSF3; OX40 Ligand/TNFSF4; TL1A/TNFSF15; TNF-alpha; Lymphotoxin-alpha/TNF-beta; TRAIL/TNFSF10; TRANCE/TNFSF11/RANK L; TWEAK/TNFSF12; and EDA-A1/Ectodysplasin A1.

The compounds of the various embodiments can be provided to an administering physician or other health care professional in the form of a kit. The kit is a package which houses a container which contains the compound(s) in a suitable pharmaceutical composition, and instructions for administering the pharmaceutical composition to a subject. The kit can optionally also contain one or more additional therapeutic agents. For example, a kit containing one or more compositions comprising compound(s) of the various embodiments in combination with one or more additional antiretroviral, antibacterial, anti-infective and/or other agents can be provided, or separate pharmaceutical compositions containing a compound of the various embodiments and additional therapeutic agents can be provided. The kit can also contain separate doses of a compound of the various embodiments for serial or sequential administration. The kit can optionally contain one or more diagnostic tools and instructions for use. The kit can contain suitable delivery devices, e.g., syringes, and the like, along with instructions for administering the compound(s) and any other therapeutic agent. The kit can optionally contain instructions for storage, reconstitution (if applicable), and administration of any or all therapeutic agents included. The kits can include a plurality of containers reflecting the number of administrations to be given to a subject.

In various embodiments, a kit for the treatment of prevention or treatment of cancer is provided that includes one of the compounds of the various embodiments and one or more antiviral agents currently employed for the treatment or prevention of HPV caused cancer, e.g., cisplatin and doxorubicin.

Other chemotherapeutic agents can also be administered with the compounds of preferred embodiments, including those that function by activating apoptosis. Suitable chemotherapeutic agents can include, but are not limited to, alkylating agents (Nitrogen mustards: such as mechlorethamine (nitrogen mustard), chlorambucil, cyclophosphamide (Cytoxan®), ifosfamide, and melphalan; Nitrosoureas: such as streptozocin, carmustine (BCNU), and lomustine; Alkyl sulfonates: busulfan; Triazines: dacarbazine (DTIC) and temozolomide (Temodar®); Ethylenimines: thiotepa and altretamine (hexamethylmelamine); platinum drugs (such as cisplatin, carboplatin, and oxalaplatin)); antimetabolites (5-fluorouracil (5-FU), 6-mercaptopurine (6-MP), Capecitabine (Xeloda®), Cytarabine (Ara-C®), Floxuridine, Fludarabine, Gemcitabine (Gemzar®), Hydroxyurea, Methotrexate, Pemetrexed (Alimta®)); anti-tumor antibiotics (anthracyclines (Daunorubicin, Doxorubicin (Adriamycin®), Epirubicin, Idarubicin); Actinomycin-D, Bleomycin, Mitomycin-C, Mitoxantrone (also acts as a topoisomerase II inhibitor)); Topoisomerase inhibitors (Topoisomerase I inhibitors including Topotecan and Irinotecan (CPT-11), Topoisomerase II inhibitors including Etoposide (VP-16), Teniposide, Mitoxantrone (also acts as an anti-tumor antibiotic)); Mitotic inhibitors (Taxanes such as paclitaxel (Taxol®) and docetaxel (Taxotere®), Epothilones: ixabepilone (Ixempra®), Vinca alkaloids: vinblastine (Velban®), vincristine (Oncovin®), and vinorelbine (Navelbine®), Estramustine (Emcyt®)); Corticosteroids (Prednisone, Methylprednisolone (Solumedrol®), Dexamethasone (Decadron®)); L-asparaginase, which is an enzyme, and the proteosome inhibitor bortezomib (Velcade®).

While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

What is claimed is:

1. A method of treating or preventing a cancer caused by a human papilloma virus, comprising administering to a subject in need thereof an effective amount of spinacine.

2. The method of claim 1, wherein the spinacine is D,L-spinacine.

3. The method of claim 1, wherein the spinacine is D-spinacine.

4. The method of claim 1, wherein the spinacine is L-spinacine.

5. The method of any one of claims 1-4, further comprising administering an effective amount of at least one chemotherapeutic agent.

6. The method of any one of claims 1-5, further comprising administering an effective amount of at least one chemotherapeutic agent selected from the group consisting of cisplatin and doxorubicin.

7. The method of any one of claims 1-6, comprising administering an effective amount of a combination of 6,7-dihydroimidazo[5,4-c]pyridine-6-carboxylic acid and spinacine.

8. The method of any one of claims 1-7, further comprising administering an effective amount of at least one TNF superfamily ligand selected from the group consisting of 4-1BB Ligand/TNFSF9, APRIL/TNFSF13, BAFF/BLyS/TNFSF13B, CD27 Ligand/TNFSF7, CD30 Ligand/TNFSF8, CD40 Ligand/TNFSF5, EDA/Ectodysplasin, EDA-A2/Ectodysplasin A2, Fas Ligand/TNFSF6, GITR Ligand/TNFSF18, LIGHT/TNFSF14, Lymphotoxin, Lymphotoxin beta/TNFSF3, OX40 Ligand/TNFSF4, TL1A/TNFSF15, TNF-alpha, Lymphotoxin-alpha/TNF-beta, TRAIL/TNFSF10, TRANCE/TNFSF11/RANK L, TWEAK/TNFSF12, and EDA-A1/Ectodysplasin A1.

9. The method of any one of claims 1-8, wherein the subject is mammalian.

10. The method of any one of claims 1-9, wherein the subject is human.

11. The method of any one of claims 1-10, wherein the cancer is selected from the group consisting of cervical cancer, head cancers, neck cancers, oral cancers, oropharyngeal cancer, anal cancer, vaginal cancer, vulvar cancer, and penile cancer.

12. The method of any one of claim 1-11, wherein the spinacine is administered parentally, intravenously, or transmebranally.

13. A method of blocking interaction between a HPV 16 E6 viral oncogene and a partner, thereby resensitizing a high-risk human papilloma virus positive cell to cell death processes, comprising contacting the cell with an effective amount of spinacine.

14. The method of claim 13, wherein the cell is mammalian.

15. The method of any one of claims 13-14, wherein the cell is human.

16. The method of any one of claims 13-15, wherein the cell is in vivo.

17. The method of any one of claims 13-16, wherein the cell is ex vivo.

18. A pharmaceutical composition comprising spinacine and a pharmaceutically acceptable excipient.

19. The pharmaceutical composition of claim 18, further comprising 6,7-dihydroimidazo[5,4-c]pyridine-6-carboxylic acid.

20. A method of treating or preventing a cancer caused by a human papilloma virus, comprising administering to a subject in need thereof an effective amount of 6,7-dihydroimidazo[5,4-c]pyridine-6-carboxylic acid.

21. The method of claim 20, further comprising administering an effective amount of at least one chemotherapeutic agent.

22. The method of any one of claims 20-21, further comprising administering an effective amount of at least one chemotherapeutic agent selected from the group consisting of cisplatin and doxorubicin.

23. The method of any one of claims 20-22, further comprising administering an effective amount of at least one TNF superfamily ligand selected from the group consisting of 4-1BB Ligand/TNFSF9, APRIL/TNFSF13, BAFF/BLyS/TNFSF13B, CD27 Ligand/TNFSF7, CD30 Ligand/TNFSF8, CD40 Ligand/TNFSF5, EDA/Ectodysplasin, EDA-A2/Ectodysplasin A2, Fas Ligand/TNFSF6, GITR Ligand/TNFSF18, LIGHT/TNFSF14, Lymphotoxin, Lymphotoxin beta/TNFSF3, OX40 Ligand/TNFSF4, TL1A/TNFSF15, TNF-alpha, Lymphotoxin-alpha/TNF-beta, TRAIL/TNFSF10, TRANCE/TNFSF11/RANK L, TWEAK/TNFSF12, and EDA-A1/Ectodysplasin A1.

24. The method of any one of claims 20-23, wherein the subject is mammalian.

25. The method of any one of claims 20-24, wherein the subject is human.

26. The method of any one of claims 20-25, wherein the cancer is selected from the group consisting of cervical cancer, head cancers, neck cancers, oral cancers, oropharyngeal cancer, anal cancer, vaginal cancer, vulvar cancer, and penile cancer.

27. The method of any one of claim 20-26, wherein the 6,7-dihydroimidazo[5,4-c]pyridine-6-carboxylic acid is administered parentally, intravenously, or transmebranally.

28. A method of blocking interaction between a HPV 16 E6 viral oncogene and a partner, thereby resensitizing a high-risk human papilloma virus positive cell to cell death processes, comprising contacting the cell with an effective amount of 6,7-dihydroimidazo[5,4-c]pyridine-6-carboxylic acid.

29. The method of claim 28, wherein the cell is mammalian.

30. The method of any one of claims 28-29, wherein the cell is human.

31. The method of any one of claims 28-30, wherein the cell is in vivo.

32. The method of any one of claims 28-31, wherein the cell is ex vivo.

33. A pharmaceutical composition comprising 6,7-dihydroimidazo[5,4-c]pyridine-6-carboxylic acid and a pharmaceutically acceptable excipient.
